(19) United States
(12) Patent Application Publication
(10) Pub. No.: US 2009/0028862 A1
(43) Pub. Date: Jan. 29, 2009

(54) Emmprin antagonists and uses thereof

(76) Inventors: Gregory M. Arndt, New South Wales (AU); Mehnaaz Lomas, New South Wales (AU); Marian T. Nakada, Malvern, PA (US); Laurent P. Rivory, New South Wales (AU); Yi Tang, Drexel Hill, PA (US); Li Yan, Berwyn, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/664,150

(22) PCT Filed: Sep. 29, 2005
(86) PCT No.: PCT/US05/34839

§ 371 Date: May 12, 2008

Related U.S. Application Data
(60) Provisional application No. 60/614,904, filed on Sep. 30, 2004.

(51) Int. Cl. A61K 39/395; A61K 31/7105; A61K 31/711; A61K 48/00; A61P 35/00

(52) U.S. Cl. 424/138.1; 514/44

(57) ABSTRACT

EMMPRIN (Extracellular Matrix Metalloproteinase Inducer) antagonists, such as antibodies, including specified portions or variants, siRNA, shRNA, antisense and DNAzymes, can be used to treat pathological processes associated with proliferative diseases, such as cancer, by specifically preventing or inhibiting the ability of proliferating tissue to develop a blood supply.

### CROSS REFERENCE TO RELATED APPLICATIONS

This application claims is a national stage of International Application Number PCT/US2005/034839, filed 29 Sep. 2005, which claims the benefit of U.S. Provisional Application No. 60/614,904, filed 30 Sep. 2004.

### FIELD OF THE INVENTION

The present invention relates to EMMPRIN (Extracellular Matrix Metalloproteinase Inducer) antagonists and a method of using EMMPRIN antagonists to treat pathological processes associated with proliferative diseases, such as cancer, by specifically preventing or inhibiting the ability of proliferating tissue to develop a blood supply. The invention more specifically relates to methods of treating such diseases by the use of EMMPRIN antagonists, such as interfering RNA, DNAzymes, and antibodies directed toward EMMPRIN, including specified portions or variants, specific for at least one protein or fragment thereof, in an amount effective to inhibit angiogenesis.

### BACKGROUND OF THE INVENTION

Angiogenesis is the process of new vessel formation. In adults, angiogenesis occurs only locally and transiently under physiological conditions such as wound healing, menstruation and pregnancy. In contrast, excessive angiogenesis occurs in more than 70 disease conditions, such as cancer, atherosclerosis, diabetic blindness, age-related macular degeneration, rheumatoid arthritis, and psoriasis. On the other hand, insufficient angiogenesis underlies diseases, such as coronary artery disease, stroke, and delayed wound healing.

Matrix metalloproteinases (MMPs), a family of more than twenty endopeptidases that are capable of cleaving all of the extracellular matrix components, play critical roles in embryonic development, tissue remodeling, wound healing, and, more specifically, in angiogenesis [Klagsbrun and Moses 1999 from reference list below]. Angiogenesis initiates as the breakdown of blood vessel basement membrane by capillary endothelial cells activated by angiogenic stimulators derived from tumors, inflammation sites, or tissues undergoing other pathological conditions.

The MMPs have different enzymatic activities and include collagenases, gelatinases and stromelysins. Many animal and human studies have found a positive correlation between the expression of MMP-1, MMP-2, MMP-7, MMP-9, MMP-11, and MT1-MMP, and tumor progression. The MMPs are thought to play a major role in tumor cell invasion and metastasis, although the exact mechanism of their action is not clear. Tumor invasion and angiogenesis both require degradation of the extracellular matrix and cleavage of certain matrix components may free growth factors. Thus, EMMPRIN-associated tumor invasion and metastasis is most likely mediated via the induction of certain MMPs.

MMPs are themselves targets of several anti-tumour therapies [Zucker, 2000]. In particular, matrix metalloproteinase inhibitors have been used including marimastat, prinomastat and BAY-12-9566. These three inhibitors were all tested in phase III clinical trials, but did not show clinical efficacy. This is probably due to the fact that MMPs are important in early aspects of cancer progression occurring before metastasis.

The activated endothelial cells express increased MMPs, which in turn, enable disseminated endothelial cells to migrate away from their parental vessels. Only after the cells escape, do they respond to various growth factors to proliferate, and eventually go through a complex differentiation process to form new vessels. Depletion of MMPs, such as MMP-2 or MMP-9, results in a significant inhibition of tumor angiogenesis, supporting the critical role of MMPs in this process.

Extracellular matrix metalloproteinase inducer (EMMPRIN) (also known as CD 147) is a 58 kDa glycoprotein, originally purified from the plasma membrane of cancer cells and was designated tumor collagenase stimulating factor (TCSF) because of its ability to stimulate collagenase-1 (MMP-1) synthesis by tumor stromal fibroblast cells [Biswas et al. 1995; Ellis et al. 1989]. It was demonstrated to be identical to the M6 antigen and human Basigin [Biswas et al. 1995]. Subsequent studies further demonstrated that EMMPRIN also induced fibroblast synthesis of MMP-2, MMP-3, as well as the membrane-type 1 MMP (MT1-MMP) and MT2-MMP that function as endogenous activators for MMP-2 [Guo et al. 1997; Kataoka et al. 1993; Sameshima et al. 2000b].

The gene encoding EMMPRIN maps to chromosome 19p13.3 in humans [Kaname, 1993] and has been well-characterized [Guo, 1998]. EMMPRIN is encoded by eight exons encompassing 10.8 kb of DNA, yielding an mRNA transcript of approximately 1.6 kb. The 5′-UTR is 37 bases in length. EMMPRIN protein is 269 amino acids in length and has a 29 amino acid long signal peptide, two extracellular immunoglobulin domains, a transmembrane domain and a carboxy-terminal cytoplasmic domain of 39 amino acids. Unlike most members of the immunoglobulin superfamily, each Ig domain of EMMPRIN is encoded by two exons rather than one.

EMMPRIN is involved in the normal induction of matrix metalloproteinases during processes, such as embryonic development and wound healing. Several clinical studies have demonstrated that the expression level of EMMPRIN in tumor tissues is significantly higher than that in peritumoral stromal tissues. These tumors include lung [Polette et al. 1997], breast [Polette et al. 1997], bladder [Javadpour and Guirguis 1992; Muraoka et al. 1993], glioma [Sameshima et al. 2000a], oral squamous cells [Bordador, 2000], Hodgkin's lymphoma, and in anaplastic large cell lymphoma [Thorns, 2002]. Examination of EMMPRIN expression in these clinical samples by a variety of means, including Northern blot, in situ hybridization and immunostaining, revealed that EMMPRIN is expressed by tumor cells, but not by the neighboring stromal cells. On the other hand, MMPs are expressed by peritumoral stromal cells. The role of EMMPRIN in tumor growth and metastasis was directly illustrated using EMMPRIN-overexpressing human breast cancer cells. MDA MB 436 cells are normally slow growing cells when they are implanted into nude mice. However, when these cells were transfected with EMMPRIN, they adopted a more aggressive growth pattern, with both accelerated growth rate and metastatic phenotypes [Zucker et al. 2001].

In addition, increased levels of EMMPRIN have been found in pigment cell lesions [van der Oord, 1997], the tissue surrounding loosened hip prostheses [Li, 1999], activated but not naïve tumor-specific T cells (measured by DNA microarray analysis) [Zhang, 2002], GM-CSF-induced differentiated monocytes (measured by Northern blot and staining) [Major, 2002], human atheromas [Major, 2002], acute vascular rejection after heart transplant [Yamani, 2002], venous leg ulcers [Norgauer, 2002], lung injury induced by mechanical stretch [Haseneen, 2003] and in the granulocytes of patients with rheumatoid arthritis.

Disorders Associated with Inappropriate Angiogenesis

Angiogenesis results from activated proliferation of endothelial cells. Neovascularization is tightly regulated, and occurs only during embryonic development, tissue remodeling, wound healing and periodic cycle of corpus luteum development (Folkman and Cotran, Relation of vascular proliferation to tumor growth, Int. Rev. Exp. Pathol.'16, 207-248 (1976)).

Endothelial cells normally proliferate much more slowly than other types of cells in the body. However, if the proliferation rate of these cells becomes unregulated, pathological angiogenesis can result. Pathological angiogenesis is involved in many diseases. For example, cardiovascular diseases, such as angioma, angiofibroma, vascular deformity, atherosclerosis, synechia and edemic sclerosis; and opthalmological diseases, such as neovascularization after cornea implantation, neovascular glaucoma, diabetic retinopathy, angiogenic corneal disease, macular degeneration, pterygium, retinal degeneration, retrolental fibroplasias, and granular conjunctivitis are related to angiogenesis. Chronic inflammatory diseases, such as arthritis; dermatological diseases, such as psoriasis, telangiectasis, pyogenic granuloma, seborrheic dermatitis, venous ulcers, acne, rosacea (acne rosacea or erythematosa), warts (verrucas), eczema, hemangiomas, lymphangiogenesis are also angiogenesis-dependent.

Vision can be impaired or lost because of various ocular diseases in which the vitreous humor is infiltrated by capillary blood. Diabetic retinopathy can take one of two forms, non-proliferative or proliferative. Proliferative retinopathy is characterized by abnormal new vessel formation (neovascularization), which grows on the vitreous surface or extends into the vitreous cavity. In advanced disease, neovascular membranes can occur, resulting in a traction retinal detachment. Vitreous hemorrhages may result from neovascularization. Visual symptoms vary. A sudden severe loss of vision can occur when there is intravitreal hemorrhage. Visual prognosis with proliferative retinopathy is more guarded if associated with severe retinal ischemia, extensive neovascularization, or extensive fibrous tissue formation. Macular degeneration, likewise takes two forms, dry and wet. In exudative macular degeneration (wet form), which is much less common, there is formation of a subretinal network of choroidal neovascularization often associated with intraretinal hemorrhage, subretinal fluid, pigment epithelial detachment, and hyperpigmentation. Eventually, this complex contracts and leaves a distinct elevated scar at the posterior pole. Both forms of age-related macular degeneration are often bilateral and are preceded by drusen in the macular region. Another cause of loss of vision related to angiogenic etiologies is damage to the iris. The two most common situations that result in the iris being pulled up into the angle are contraction of a membrane such as in neovascular glaucoma in patients with diabetes or central retinal vein occlusion or inflammatory precipitates associated with uveitis pulling the iris up into the angle (Ch. 99. The Merck Manual 17^{th} Ed. 1999).

Rheumatoid arthritis, an inflammatory disease, also results in inappropriate angiogenesis. The growth of vascular endothelial cells in the synovial cavity is activated by the inflammatory cytokines, and results in cartilage destruction and replacement with pannus in the articulation (Koch A K, Polyerini P J and Leibovich S J, Arth; 15 Rhenium, 29, 471-479 (1986); Stupack D G, Storgard C M and Cheresh D A, Braz. J. Med. Biol. Res., 32, 578-581 (1999); Koch A K, Arthritis Rheum, 41, 951 962 (1998)).

Psoriasis is caused by uncontrolled proliferation of skin cells. Fast growing cells require sufficient blood supply, and abnormal angiogenesis is induced in psoriasis (Folkman J., J. Invest. Dermatol., 59, 40-48 (1972)).

There is now considerable evidence that tumor growth and cancer progression requires angiogenesis, the formation of new blood vessels in order to provide tumor tissue with nutrients and oxygen, to carry away waste products and to act as conduits for the metastasis of tumor cells to distant sites (Folkman, et al. N Engl J Med 285: 1181-1186, 1971 and Folkman, et al. N Engl J Med 333: 1757-1763, 1995).

A number of factors are involved in processes and events leading to angiogenesis: cell adhesion molecules, integrins, vascular endothelial growth factor (VEGF), TNFalpha, bFGF, and cytokines including IL-6 and IL-12. For example, the closely related but distinct integrins αVβ3 and αVβ5 have been shown to mediate independent pathways in the angiogenic process. An antibody generated against αVβ3 blocked basic fibroblast growth factor (bFGF) induced angiogenesis, whereas an antibody specific to αVβ5 inhibited vascular endothelial growth factor (VEGF) induced angiogenesis (Eliceiri, et al., J. Clin. invest. 103: 1227-1230 (1999); Friedlander et al., Science 270: 1500-1502 (1995)). IL-6 is elevated in tissues undergoing angiogenesis and can induce VEGF in A431 cells, a human epidermoid carcinoma cell line (Cohen, et al. J. Biol. Chem. 271: 736-741, 1996).

Thus, angiogenesis is known to be a contributing factor in a number of pathological conditions, including the ability of tumors to grow and metastasize, disorders of the eye including retinopathies, and disorders of the skin including Kaposi's Sarcoma. While numerous factors have been shown to be associated with these processes, it has not heretofore been demonstrated that EMMPRIN directly stimulates VEGF production, stimulates endothelial cells, in addition to local fibroblast cells, to express MMPs and therefore facilitate tumor angiogenesis, growth, invasion and metastasis.

Gene expression can be modulated in several different ways including by the use of siRNAs, shRNAs, antisense molecules and DNAzymes (collectively referred to as “nucleic acid based antagonists”. SiRNAs and shRNAs both work via the RNAi pathway and have been successfully used to suppress the expression of genes. RNAi was first discovered in plants by Fire and Mello and is thought to be a way for plant cells to combat infection with RNA viruses. In this pathway, the long dsRNA viral product is processed into smaller fragments of 21-25 bp in length by a DICER-like enzyme and then the double-stranded molecule is unwound and loaded into the RNA induced silencing complex (RISC). A similar pathway has been identified in mammalian cells with the notable difference that the dsRNA molecules must be smaller than 50 bp in length in order to avoid the induction of the so-called interferon response, which is not gene specific and leads to the global shut down of protein synthesis in the cell.

Synthetic siRNAs can be designed to specifically target one gene and they can easily be delivered to cells in vitro or in vivo. SiRNAs can be incorporated into DNA expressed hairpin structures (shRNA). The shRNA have the advantage of being incorporated into the cells' genome and then being replicated during every mitotic cycle.

DNAzymes have also been used to modulate gene expression. DNAzymes are catalytic DNA molecules that cleave single-stranded RNA. They are highly selective for the target RNA sequence and as such can be used to down-regulate specific genes through targeting of the messenger RNA.

### SUMMARY OF THE INVENTION

The present invention relates to EMMPRIN antagonists and a method of using EMMPRIN antagonists, including nucleic acid based antagonists (siRNA, shRNA, antisense, and DNAzymes) directed toward EMMPRIN, and specified portions or variants thereof specific for at least one EMMPRIN protein or fragment thereof, or an EMMPRIN transcript or fragment or variant thereof, to inhibit angiogenesis in disease conditions associated with abnormal angiogenesis. Such EMMPRIN antagonists can act through their ability to prevent the ability of EMMPRIN from stimulating MMP expression by microvascular endothelial cells, the cells involved in angiogenesis, in a dose-dependent fashion. Secondly, such antagonists can act by limiting EMMPRIN induction of VEGF in the local environment, thereby reducing the angiogenic potential of the tissue. By interfering with angiogenesis, such antagonists can prevent events associated with the initiation or progression of cancer tissue including events involved with angiogenesis and the metastatic spread of cancer. Based on the aforementioned action of the EMMPRIN antagonists of the invention, these antagonists can be best described as anti-angiogenic EMMPRIN antagonists.

Thus, the present invention demonstrates for the first time that EMMPRIN can directly stimulate MMP-1 expression by microvascular endothelial cells, the cells involved in angiogenesis, in a dose-dependent fashion. This stimulation is specifically inhibited by function-blocking EMMPRIN antagonists, such as monoclonal antibodies or nucleic acid based antagonists. Since MMPs are essential for angiogenesis, such EMMPRIN antagonists can be useful as therapeutics for such diseases as cancer, diabetic blindness, age-related macular degeneration, rheumatoid arthritis, and psoriasis.

In one embodiment, the EMMPRIN antagonist is an siRNA molecule, an shRNA molecule, or a DNAzyme capable of preventing the production of EMMPRIN by cells.

In a particular embodiment, the EMMPRIN antagonist is an antibody that specifically binds EMMPRIN. A particular advantage of such antibodies is that they are capable of binding EMMPRIN in a manner that prevents its action systemically. The method of the present invention thus employs antibodies having the desirable neutralizing property which makes them ideally suited for therapeutic and preventative treatment of metastatic disease states associated with various forms of cancer in human or nonhuman patients. Accordingly, the present invention is directed to a method of treating a disease or condition which is dependent on angiogenesis in a patient in need of such treatment which comprises administering to the patient an amount of a neutralizing EMMPRIN antibody to inhibit angiogenesis.

### BRIEF DESCRIPTION OF THE DRAWINGS

FIG. 1 is a schematic illustration of the central role of EMMPRIN in diseases involving abnormal angiogenesis.

FIG. 2 shows that recombinant EMMPRIN dose-dependently stimulated MMP-1 production by HMVEC-L cells.

FIG. 3 shows inhibition of EMMPRIN-induced MMP-1 production in HMVEC-L cells by a neutralizing anti-EMMPRIN monoclonal antibody.

FIG. 4A shows a bar graph demonstrating the relative level of HUVEC cell migration induced by conditioned medium derived from various MDA-MB-231 cell constructs.

FIG. 4B shows a bar graph demonstrating the relative reduction in the migration of endothelial cells induced by WT cells in the presence of increasing concentrations of anti-VEGF antibody.

FIG. 5A is a bar graph showing the average final tumor weights of tumors produced by MDA MB231 human breast tumor cells manipulated to express greater (S1-3) or lesser (AS1-5) (AS2-5) amounts of EMMPRIN than normal (WT) or Vector control cells.

FIG. 5B is a micrograph showing the difference in angiogenic structures between tumors produced by implantation of mice with WT versus S1-3 cells.

FIG. 5C is a set of bar graphs showing the amount of human VEGF (left panel) and mouse VEGF (right panel) in tumors produced by MDA MB231 human breast tumor cell types.

FIG. 6A is a bar graph showing the amount of human EMMPRIN in tissue extracts from xenograft tumors derived from WT, Vector control, S1-3, or AS EMMPRIN engineered human tumor cells.

FIG. 6B is a photo of a zymography gel showing MMP expression profile in tissue extracts from the same tumors where 10 μg of total protein was added to each lane.

FIG. 6C is a bar graph showing the quantitative determination of human and mouse MMP-2 levels in xenograft tumors.

FIG. 6D is a pair of bar graphs showing quantitative determination of human (left panel) and mouse (right panel) MMP-9 levels in xenograft tumors.

FIGS. 7A-C show photographs demonstrating increased angiogenesis evidenced by numerous new capillary blood vessels in tumors derived from sense cells expressing EMMPRIN, but not in tumors derived from WT OR AS cells.

FIG. 8 shows photographs of tumors after immunohistochemical analysis of MMP, VEGF, EMMPRIN: H&E staining of MDA-MB-231 xenograft tumors; Mouse MMP-9 staining; Mouse EMMPRIN staining; and Blood vessel staining with anti-CD31 antibodies. The left panels are Vector control tumors; the right panels are S1-3 tumors.

FIGS. 9A-E show suppression of EMMPRIN expression on the surface of MDA MB 231 cells following transient transfection with specific siRNAs (60 nM).

FIG. 10 shows EMMPRIN expression on MDA MB 231 cells stably transfected with pSilencer 2.1 shEMM1.

FIGS. 11A and B show EMMPRIN expression in stable pSilencer clones generated in the MDA MB 231 and MDA MB 435S-GFP cell lines measured by Western analysis.

FIGS. 12A and B show EMMPRIN expression in stable pSilencer clones generated in the MDA MB 231 and MDA MB 435S-GFP cell lines measured by northern analysis.

FIGS. 13A and B show the results of an EMMPRIN-specific Qzyme assay.

FIGS. 14A-E show overexpression of EMMPRIN on the surface of MDA MB 231 cells after transfection with various EMMPRIN overexpression constructs as measured by antibody staining and flow cytometry.

FIGS. 15A-D show EMMPRIN expression on the surface of various cell lines measured by antibody staining and flow cytometry.

FIGS. 16A-F show EMMPRIN expression on the surface of various colorectal cancer cell lines.

FIG. 17 shows the inhibition of EMMPRIN levels and VEFG production by shEMM6, C1 and shEMM1, C8 clones.

### DETAILED DESCRIPTION OF THE INVENTION

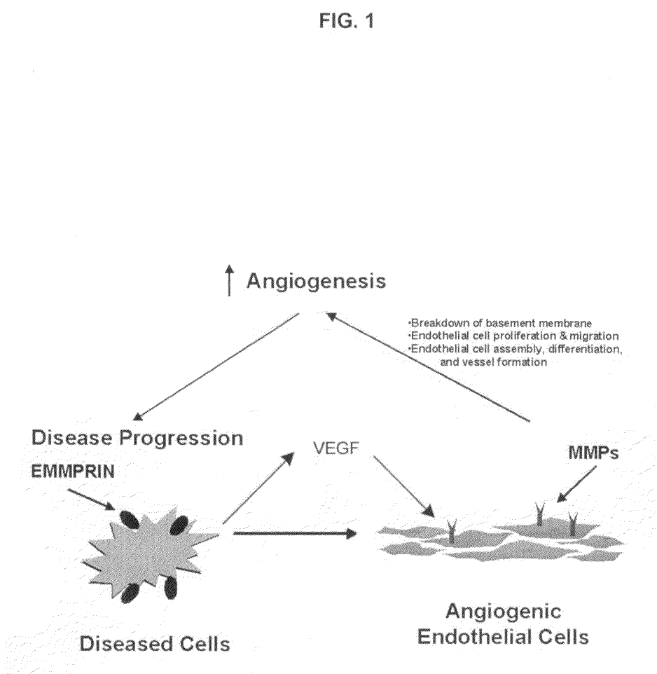

EMMPRIN expressed by cells in diseased tissues directly stimulates neighboring endothelial cells, which results in an increase in MMP expression, i.e., MMP-1 (See FIG. 1). These MMPs, in turn, mediate the breakdown of basement membrane of existing blood vessels; promote endothelial cells to migrate away from parental vessels; stimulate the expression and release of angiogenic growth factors; enable endothelial cells to respond to angiogenesis stimulatory factors leading to cell proliferation; and facilitate the remodeling of extracellular matrix for endothelial cell differentiation and assembly of new vessels. All of these changes lead to an increase in angiogenesis and further contribute to the overall disease progression.

The anti-angiogenic EMMPRIN antagonists of the invention are useful in inhibiting and preventing angiogenesis in so far as they block the stimulatory effects of EMMPRIN on endothelial cells, reduce VEGF production by endothelial cells, reduce endothelial cell division, decrease endothelial cell migration, and impair the activity of the proteolytic enzymes secreted by the endothelium. A number of pathologies, including various forms of solid primary tumors and the metastases, lesions of the eye and disorders of the skin, are improved by treatment with EMMPRIN antagonists in the method of the present invention.

Cancer

Both benign and malignant tumors, including various cancers, such as, cervical, anal and oral cancers, stomach, colon, bladder, rectal, liver, pancreatic, lung, breast, cervix uteri, corpus uteri, ovary, prostate, testis, renal, brain/cns (e.g., gliomas), head and neck, eye or ocular, throat, skin melanoma, acute lymphocytic leukemia, acute myelogenous leukemia, Ewing's Sarcoma, Kaposi's Sarcoma, basal cell carinoma and squamous cell carcinoma, small cell lung cancer, choriocarcinoma, rhabdomyosarcoma, angiosarcoma, hemangioendothelioma, Wilms Tumor, neuroblastoma, mouth/pharynx, esophageal, larynx, kidney and lymphoma, among others may be treated using EMMPRIN antagonists, such as anti-EMMPRIN antibodies or nucleic acid based antagonists of the present invention. In addition, conditions, such as neurofibromatosis, tuberous sclerosis (each of which conditions produces benign tumors of the skin), hemangiomas and lymphangiogenesis, among others, may be treated effectively with EMMPRIN antagonists according to the present invention

A secondary tumor, a metastasis, is a tumor which originated in a primary site elsewhere in the body, but has now spread to a distant organ. The common routes for metastasis are direct growth into adjacent structures, spread through the vascular or lymphatic systems, and tracking along tissue planes and body cavities with, for example, peritoneal fluid or cerebrospinal fluid. Secondary hepatic tumors are one of the most common causes of death in cancer patients and are by far and away the most common form of liver tumor. Although virtually any malignancy can metastasize to the liver, tumors which are most likely to spread to the liver include: cancer of the stomach, colon, and pancreas; melanoma; tumors of the lung, oropharynx, and bladder; Hodgkin's and non-Hodgkin's lymphoma; tumors of the breast, ovary, and prostate. Secondary lung, brain, and bone tumors are common to advanced stage breast, prostate and lung cancers. Any cancer may metastasize to bone, but metastases from carcinomas are the most common, particularly those arising in the breast, lung, prostate, kidney, and thyroid. Carcinoma of the lung is very commonly accompanied by hematogenous metastatic spread to the liver, brain, adrenals, and bone and may occur early, resulting in symptoms at those sites before obvious pulmonary symptoms. Metastases to the lungs are common from primary cancers of the breast, colon, prostate, kidney, thyroid, stomach, cervix, rectum, testis, and bone and from melanoma. Each one of the above-named secondary tumors may be treated by the antagonists of the present invention.

In addition to tumors, numerous other non-tumorigenic angiogenesis-dependent diseases which are characterized by the abnormal growth of blood vessels, may also be treated with the anti-angiogenic EMMPRIN antagonists of the present invention.

Representative examples of such non-tumorigenic angiogenesis-dependent diseases include corneal neovascularization, hypertrophic scars and keloids, proliferative diabetic retinopathy, rheumatoid arthritis, arteriovenous malformations (discussed above), atherosclerotic plaques, delayed wound healing, hemophilic joints, nonunion fractures, Osler-Weber syndrome, psoriasis, pyogenic granuloma, scleroderma, tracoma, menorrhagia (discussed above) and vascular adhesions.

Angiogenic Conditions of the Eyes

The cornea is a tissue which normally lacks blood vessels. In certain pathological conditions, however, capillaries may enter the cornea from the pericorneal vascular plexus of the limbus. When the cornea becomes vascularized, it also becomes clouded, resulting in a decline in the patient's visual acuity. Visual loss may become complete if the cornea completely opacitates.

Blood vessels can enter the cornea in a variety of patterns and depths, depending upon the process which incites the neovascularization. These patterns have been traditionally defined by ophthalmologists in the following types: pannus trachomatosus, pannus leprosus, pannus phylctenulosus, pannus degenerativus, and glaucomatous pannus. The corneal stroma may also be invaded by branches of the anterior ciliary artery (called interstitial vascularization) which causes several distinct clinical lesions: terminal loops, a “brush-like” pattern, an umbel form, a lattice form, interstitial arcades (from episcleral vessels), and aberrant irregular vessels.

Corneal neovascularization can result from corneal ulcers. A wide variety of etiologies may produce corneal ulcers including, for example, corneal infections (trachoma, herpes simplex keratitis, leishmaniasis and onchocerciasis), immunological processes (graft rejection and Stevens-Johnson's syndrome), alkali burns, trauma, inflammation (of any cause), toxic and Vitamin A or protein deficiency states, and as a complication of wearing contact lenses.

While the cause of corneal neovascularization may vary, the response of the cornea to the insult and the subsequent vascular ingrowth is similar regardless of the cause. Several angiogenic factors are likely involved in this process, many of which are products of the inflammatory response. Indeed, neovascularization of the cornea appears to only occur in association with an inflammatory cell infiltrate, and the degree of angiogenesis is proportional to the extent of the inflammatory reaction. Corneal edema further facilitates blood vessel ingrowth by loosening the corneal stromal framework through which the capillaries grow.

Topical therapy with an EMMPRIN antagonist may also be useful prophylactically in corneal lesions which are known to have a high probability of inducing an angiogenic response (such as chemical burns). In these instances, the treatment, likely in combination with steroids, may be instituted immediately to help prevent subsequent complications.

Such methods may also be utilized in a similar fashion to prevent capillary invasion of transplanted corneas. Use in combination with a steroid is also contemplated.

Neovascular glaucoma is a pathological condition wherein new capillaries develop in the iris of the eye. The angiogenesis usually originates from vessels located at the pupillary margin, and progresses across the root of the iris and into the trabecular meshwork. Fibroblasts and other connective tissue elements associate with the capillary growth and a fibrovascular membrane develops which spreads across the anterior surface of the iris eventually forming a scar. The scar formation prevents adequate drainage of aqueous humor resulting in an increase in intraocular pressure that may result in blindness.

Neovascular glaucoma generally occurs as a complication of diseases in which retinal ischemia is predominant. In particular, about one third of the patients with this disorder have diabetic retinopathy. Other causes include chronic retinal detachment, end-stage glaucoma, carotid artery obstructive disease, retrolental fibroplasia, sickle-cell anemia, intraocular tumors, and carotid cavernous fistulas.

Angiogenic Conditions of the Skin

Within another aspect of the present invention, methods are provided for treating hypertrophic scars and keloids, comprising the step of administering one of the above-described anti-angiogenic compositions to a hypertrophic scar or keloid.

Healing of wounds and scar formation occurs in three phases: inflammation, proliferation, and maturation. The first phase, inflammation, occurs in response to an injury which is severe enough to cause tissue damage and vascular leaking. During this phase, which lasts 3 to 4 days, blood and tissue fluid form an adhesive coagulum and fibrinous network which serves to bind the wound surfaces together. This is then followed by a proliferative phase in which there is ingrowth of capillaries and connective tissue from the wound edges, and closure of the skin defect. Finally, once capillary and fibroblastic proliferation has ceased, the maturation process begins wherein the scar contracts and becomes less cellular, less vascular, and appears flat and white. This final phase may take between 6 and 12 months.

Overproduction of connective tissue at the wound site causes a persistently cellular and possible red and raised scar to be formed. If the scar remains within the boundaries of the original wound it is referred to as a hypertrophic scar, but if it extends beyond the original scar and into the surrounding tissue, the lesion is referred to as a keloid. Hypertrophic scars and keloids are produced during the second and third phases of scar formation. Several wounds are particularly prone to excessive endothelial and fibroblastic proliferation, including burns, open wounds, and infected wounds. With hypertrophic scars, some degree of maturation occurs and gradual improvement occurs. In the case of keloids however, an actual tumor is produced which can become quite large. Spontaneous improvement in such cases rarely occurs. Administration of an EMMPRIN antagonist, e.g., antibody, in the method of the present invention to inhibit angiogenesis in such conditions can thus inhibit the formulation of such keloid scars.

Anti-Angiogenic Combinations with EMMPRIN Antagonists

Angiogenesis is characterized by the invasion, migration and proliferation of smooth muscle and endothelial cells. The αvβ3 integrin (also known as the vitronectin receptor) is known to play a role in various conditions or disease states including tumor metastasis, solid tumor growth (neoplasia), osteoporosis, Paget's disease, humoral hypercalcemia of malignancy, angiogenesis, including tumor angiogenesis, retinopathy, including macular degeneration, arthritis, including rheumatoid arthritis, periodontal disease, psoriasis and smooth muscle cell migration (e.g., restenosis).

The adhesion receptor integrin αvβ3 binds vitronectin, fibrinogen, von Willebrand Factor, laminin, thrombospondin, and other like ligands. It was identified as a marker of angiogenic blood vessels in chick and man and plays a critical role in angiogenesis or neovascularization. Antagonists of αvβ3 inhibit this process by selectively promoting apoptosis of cells in neovasculature. Therefore, αvβ3 antagonists would be useful therapeutic targets for treating such conditions associated with neovascularization (Brooks et al., Science, Vol. 264, (1994), 569-571). Additionally, tumor cell invasion occurs by a three step process: (1) tumor cell attachment to extracellular matrix; (2) proteolytic dissolution of the matrix; and (3) movement of the cells through the dissolved barrier. This process can occur repeatedly and can result in metastases at sites distant from the original tumor. The αvβ3 integrin has been shown to play a role in tumor cell invasion as well as angiogenesis.

As the antagonists of αvβ3 and neutralizing anti-EMMPRIN antibodies both target neovasculature but act through different mechanisms, the combination of anti-integrin antibodies (or other antagonists) with anti-EMMPRIN antibodies (or other antagonists) should result in a particularly potent and effective combination therapy with little normal tissue toxicity. Thus, in one embodiment of the present invention, there is provided a method of treating a disease or condition associated with angiogenesis which comprises administering a combination of an integrin antagonist and an EMMPRIN antagonist to inhibit angiogenesis in a patient in need of such treatment. Other antibodies which selectively bind integrins or integrin subunits, especially those that bind the alphaV subunit, are disclosed in U.S. Pat. Nos. 5,985,278 and 6,160,099. Mabs that inhibit binding of alphaVbeta3 to its natural ligands containing the tripeptide argininyl-glycyl-aspartate (RGD) are disclosed in U.S. Pat. No. 5,766,591 and WO/0078815.

A preferred combination of antibodies is the anti-alphaVbeta3 and anti-alphaVbeta5 Mab described in applicant's co-pending application U.S. Ser. No. 09/920,267 and an anti-EMMPRIN antibody, as disclosed herein. Both of the foregoing applications are incorporated by reference into the present application and form part of the disclosure hereof. In accordance with the invention, other known anti-angiogenesis agents, such as thalidomide, may also be employed in combination with an anti-EMMPRIN antibody. Furthermore, the present invention contemplates administering an EMMPRIN antagonist, e.g., a nucleic acid based antagonist, along with one or more anti-EMMPRIN antibodies or other EMMPRIN antagonists such that one or more of the antagonist or antibody can be used for targeting/delivery and one or more of the antagonist or antibody can be used for its anti-EMMPRIN or anti-angiogenesis activity.

Methods of Evaluating Anti-Angiogenic Activity

Widely accepted functional assays of angiogenesis and, hence, anti-angiogenic agents are the chick chorio-allantoic membrane assay (CAM) assay and the corneal micropocket assay of neovascularization.

For the CAM assay, fertilized chick embryos are removed from their shell on day 3 (or 4) and incubated in a Petri dish in high humidity and 5% CO₂. On day 6, a methylcellulose disc (10 microL) containing the test substance is implanted on the chorioallantoic membrane. The embryos are examined 48 hours later, and if a clear avascular zone appears around the methylcellulose disc, the diameter of that zone is measured. The larger the zone, the more effective the antibody. India ink can be injected into the heart of some embryos just before formalin fixation so that vessels are visible near the edge of the avascular zone in histological sections. Histologic cross-sections of the chorioallantoic are examined to determine whether the test substance prevents normal development of the capillaries. This method, described in U.S. Pat. No. 5,001,116 which is also specifically incorporated herein by reference, showed the test useful in the selection of anti-angiogenic compounds or combinations of compounds.

The corneal micropocket assay of neovascularization may be practiced using rat or rabbit corneas. This in vivo model is widely accepted as being generally predictive of clinical effect, as described in many review articles and papers such as O'Reilly et. al. Cell 79: 315-328.

Briefly, a plug or pellet containing the recombinant bFGF (Takeda Pharmaceuticals-Japan) is implanted into corneal micropockets of each eye of an anesthetized female New Zealand white rabbit, 2 mm from the limbus followed by topical application of erythromycin ointment onto the surface of the cornea. The animals are dosed with the test compounds and examined with a slit lamp every other day by a corneal specialist. Various mathematical models are utilized to determine the amount of vascularized cornea and this formula was found to provide the most accurate approximation of the area of the band of neovascularization that grows towards the pellet. The method may also be practiced using rats.

In the present invention, the corneal micropocket assay may be used to demonstrate the anti-angiogenesis effect of EMMPRIN antagonists. This is evidenced by a significant reduction in angiogenesis, as represented by a consistently observed and preferably marked reduction in the number of blood vessels within the cornea.

Endothelial and Non-Endothelial Cell Proliferation

It is important to establish which cell types are involved in the angiogenic processes specific for tumor vascularization. Tumor vessels are generally primitive, that is, contain only endothelial cells. Other cell types found in more mature vessels include: smooth muscle cells, retinal pigment epithelial cells, fibroblasts, and epithelial cells, as well as tumor cells such as hemangioendothelioma cells or carcinoma cells. One example of an angiogenesis inhibitor that specifically inhibits endothelial cell proliferation is ANGIOSTATIN® protein. (O'Reilly et al., 1994 supra).

Various representative cell lines are available for testing. Bovine aortic smooth muscle (SMC), bovine retinal pigment epithelial (RPE), mink lung epithelial (MLE), Lewis lung carcinoma (LLC), and EOMA hemangioendothelioma cells and 3T3 fibroblasts. For the proliferation assays, cells are washed with PBS and dispersed in a 0.05% solution of trypsin. Optimal conditions for the cell proliferation assays are established for each different cell type. Generally, cells are trypsinized and re-seeded in growth medium in the presence and absence of EMMPRIN and anti-EMMPRIN neutralizing Mab. After approximately 72 hours, the change in cell number is assessed by using a vital stain, such as a tetrazolium dye, or by LDH release (Promega, Madison Wis.) or by individual cell counting.

EMMPRIN Antagonists

As used herein, the term “EMMPRIN antagonists” refers to substances which inhibit or neutralize the angiogenic activity of EMMPRIN. Such antagonists accomplish this effect in a variety of ways. One class of EMMPRIN antagonists will bind to EMMPRIN protein with sufficient affinity and specificity to neutralize the angiogenic effect of EMMPRIN. Included in this class of molecules are antibodies and antibody fragments (such as for example, F(ab) or F(ab′)₂ molecules). Another class of EMMPRIN antagonists are fragments of EMMPRIN protein, muteins or small organic molecules, i.e., peptidomimetics, that will bind to EMMPRIN or EMMPRIN binding partners, thereby inhibiting the angiogenic activity of EMMPRIN. The EMMPRIN antagonist may be of any of these classes as long as it is a substance that inhibits EMMPRIN angiogenic activity. EMMPRIN antagonists include EMMPRIN antibody, EMMPRIN receptor antibody, modified EMMPRIN, antisense EMMPRIN and partial peptides of EMMPRIN or EMMPRINR. Another class of EMMPRIN antagonists include nucleic acid based siRNAs, shRNAs, antisense molecules and DNAzymes targeting the EMMPRIN gene sequence as known in the art and disclosed herein.

Anti-EMMPRIN Antibodies

Neutralizing antibodies to soluble factors that mediate inflammation and tumor proliferation, such as TNFalpha, have proved to highly effective therapeutics. REMICADE (infliximab) sold by Centocor, Malvern, Pa., an anti-TNFalpha MAb is prescribed for RA and Crohn's Disease and RITUXAN (rituximab), an anti-CD20 Mab sold by Genentech, San Bruno, Calif., is used to treat B-cell lymphoma. “Neutralizing” Mabs not only bind their target but also inhibit its biological activity, usually by preventing its interaction with its cognate cell surface receptor. In certain cases, the target protein will comprise more than one active domain and exhibit multiple actions due to binding to more than one ligand or receptor. EMMPRIN is such a molecule and exhibits two immunoglobulin-like domains in the extracellular portion of the molecule, the Ig-like C2-type domain from aa 22-103 of basigin isoform 2 (NCBI accession # NP_{—}940991) domain and the Ig-like V-type domain at 105-199 of the same isoform (Biswas, Zhang, DeCastro, Guo, Nakamura, Kataoka and Nabeshima, (1995), Cancer Res 55: 434-9). Monoclonal antibodies raised to EMMPRIN from cancer cells are capable of inhibiting EMMPRIN-induced MMP production in fibroblast cells, indicating neutralizing activity (Ellis, Nabeshima and Biswas, (1989), Cancer Res 49: 3385-91). These antibodies were subsequently shown to bind to EMMPRIN in the region 34-99 which lies within the C2-type domain. In contrast, CBL1, a murine IgM, anti-human lymphoblastoid monoclonal antibody that was raised in Balb/c mice immunized with the T cell acute lymphoblastic leukemia cell line (T-ALL) CEM. The latter MAb has been tested clinically in patients with graft versus host disease (Heslop, H. et al. (1995) Lancet 346: 805-806). WO9945031 teaches that antibodies with activities similar to CBL1 share a consensus binding sequence located in a region more C-terminal than the V-type domain, that is RVSR (residues 201-204 of NP_{—}940991) and of a panel of MAbs made to the extracellular domain of EMMPRIN only one, designated M-6/6, is capable of inhibiting OKT3-induced T-cell activation and binds to a region in the C2-type domain Koch, C. et al. (1999) Internat. Immunol. 11: 777-786; Staffler, G. et al. (2003) J. Immunol. 171: 1707-1714). Therefore, selection of a uniquely anti-angiogenic anti-EMMPRIN Mab can be achieved by using a specific set of in vitro assays as screening tools.

Any of the anti-EMMPRIN antibodies known in the art which are anti-angiogenic EMMPRIN antagonists may be employed in the method of the present invention. Murine monoclonal antibodies to EMMPRIN are known as in, for example, in Ellis et al, 1989 supra and Koch, et al. 1999 Internat. Immunol. 11 (5): 777-786.

Accordingly, as used herein, an “EMMPRIN antibody,” “anti-EMMPRIN antibody,” “anti-EMMPRIN antibody portion,” or “anti-EMMPRIN antibody fragment” and/or “anti-EMMPRIN antibody variant” and the like include any protein or polypeptide containing molecule that comprises at least a portion of an immunoglobulin molecule, such as, but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an EMMPRIN binding protein derived from a EMMPRIN protein or peptide, which can be incorporated into an antibody for use in the present invention. Such antibody optionally further affects a specific ligand, such as but not limited to where such antibody modulates, decreases, increases, antagonizes, agonizes, mitigates, alleviates, blocks, inhibits, abrogates and/or interferes with EMMPRIN angiogenic activity, in vitro, in situ and/or in vivo. As a non-limiting example, a suitable anti-EMMPRIN antibody, specified portion or variant of the present invention can bind at least one EMMPRIN protein or peptide, or specified portions, variants or domains thereof. A suitable anti-EMMPRIN antibody, specified portion, or variant affects EMMPRIN angiogenic function in a variety of ways, such as but not limited to, RNA, DNA or protein synthesis, EMMPRIN release, EMMPRIN receptor signaling, EMMPRIN receptor binding, EMMPRIN production and/or synthesis. The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian EMMPRIN. For example, antibody fragments capable of binding to EMMPRIN or portions thereof, including, but not limited to Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, ImmunologyT).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CH1 domain and/or hinge region of the heavy chain. The various portions of antibodies can be joined together chemically by conventional techniques, or can be prepared as a contiguous protein using genetic engineering techniques.

The anti-EMMPRIN antibody may be a primate, rodent, or human antibody or a chimeric or humanized antibody. As used herein, the term “human antibody” refers to an antibody in which substantially every part of the protein (e.g., CDR, framework, CL, CH domains (e.g., CH1, CH2, CH3), hinge, (VL, VH)) is substantially non-immunogenic in humans, with only minor sequence changes or variations, and/or is engineered to, derived from, or contains known human antibody components. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies of the invention can include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, a Fv can comprise a linker peptide, such as 2 to about 8 glycine or other amino acid residues, which connects the variable region of the heavy chain and the variable region of the light chain. Such linker peptides are considered to be of human origin.

Bispecific, heterospecific, heteroconjugate or similar antibodies can also be used that are monoclonal, preferably human or humanized, antibodies that have binding specificities for at least two different antigens. In the present case, one of the binding specificities is for at least one EMMPRIN protein, the other one is for any other antigen. Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature 305:537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed, e.g., in WO 93/08829, U.S. Pat. Nos. 6,210,668, 6,193,967, 6,132,992, 6,106,833, 6,060,285, 6,037,453, 6,010,902, 5,989,530, 5,959,084, 5,959,083, 5,932,448, 5,833,985, 5,821,333, 5,807,706, 5,643,759, 5,601,819, 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986), each entirely incorporated herein by reference.

Anti-EMMPRIN antibodies useful in the methods and compositions of the present invention can optionally be characterized by high affinity binding to EMMPRIN and optionally and preferably having low toxicity. In particular, an antibody, specified fragment or variant of the invention, where the individual components, such as the variable region, constant region and framework, individually and/or collectively, optionally and preferably possess low immunogenicity, is useful in the present invention. The antibodies that can be used in the invention are optionally characterized by their ability to treat patients for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), entirely incorporated herein by reference).

Suitable antibodies include those that compete for binding to human EMMPRIN with the commercially available monoclonal antibody CD147-RDI/clone UM-8D6 (Research Diagnostics, Inc., Flanders, N.J.).

EMMPRIN Antagonists in the Form of siRNA, shRNA, and DNAzymes

A therapeutic targeting the inducer of several MMPs may provide better chances of success. Gene expression can be modulated in several different ways including by the use of siRNAs, shRNAs, antisense molecules and DNAzymes. Synthetic siRNAs, shRNAs, and DNAzymes can be designed to specifically target one or more genes and they can easily be delivered to cells in vitro or in vivo.

Compositions and their Uses

In accordance with the invention, the neutralizing EMMPRIN antagonists, such as monoclonal antibodies or nucleic acid based antagonists, described herein can be used to inhibit angiogenesis and thus prevent or impair tumor growth and prevent or inhibit metastases. Additionally, such antagonists can be used to inhibit angiogenic inflammatory diseases amenable to such treatment, which may include, but are not limited to, rheumatoid arthritis, diabetic retinopathy, psoriasis, and macular degeneration. The individual to be treated may be any mammal and is preferably a primate, a companion animal which is a mammal and, most preferably, a human patient. The amount of antagonist administered will vary according to the purpose it is being used for and the method of administration.

The anti-angiogenic EMMPRIN antagonists may be administered by any number of methods that result in an effect in tissue in which angiogenesis is desired to be prevented or halted. Further, the anti-angiogenic EMMPRIN antagonists need not be present locally to impart an anti-angiogenic effect, therefore, they may be administered wherever access to body compartments or fluids containing EMMPRIN is achieved. In the case of inflamed, malignant, or otherwise compromised tissues, these methods may include direct application of a formulation containing the antagonists. Such methods include intravenous administration of a liquid composition, transdermal administration of a liquid or solid formulation, oral, topical administration, or interstitial or inter-operative administration. Administration may be affect by the implantation of a device whose primary function may not be as a drug delivery vehicle as, for example, a vascular stent.

In particular, within one aspect of the present invention methods are provided for treating corneal neovascularization (including corneal graft neovascularization), comprising the step of administering a therapeutically effective amount of an anti-angiogenic EMMPRIN antagonist of the invention directly to the cornea or systemically to the patient, such that the formation of blood vessels is inhibited.

Within another aspect of the present invention methods are provided for treating neovascular glaucoma, comprising the step of administering a therapeutically effective amount of an antagonist, such as an anti-angiogenic neutralizing anti-EMMPRIN antibody, directly to the eye or systemically to the patient, such that the formation of blood vessels is inhibited.

In another embodiment of the present invention either an anti-angiogenic EMMPRIN antagonist of the invention alone, or in combination with another anti-angiogenic agent are directly injected into a hypertrophic scar or keloid in order to prevent the progression of these lesions. This therapy is of particular value in the prophylactic treatment of conditions which are known to result in the development of hypertrophic scars and keloids such as burns. Therapy may be effective when begun after the proliferative phase has had time to progress (approximately 14 days after the initial injury), but before hypertrophic scar or keloid development.

Administration may also be oral or by local injection into a tumor or tissue but generally, a monoclonal antibody is administered intravenously. Generally, the dosage range is from about 0.05 mg/kg to about 12.0 mg/kg. This may be as a bolus or as a slow or continuous infusion which may be controlled by a microprocessor controlled and programmable pump device.

Alternatively, DNA encoding preferably a fragment of a monoclonal antibody may be isolated from hybridoma cells and administered to a mammal. The DNA may be administered in naked form or inserted into a recombinant vector, e.g., vaccinia virus, in a manner which results in expression of the DNA in the cells of the patient and delivery of the antibody.

The monoclonal antibody used in the method of the present invention may be formulated by any of the established methods of formulating pharmaceutical compositions, e.g. as described in Remington's Pharmaceutical Sciences, 1985. For ease of administration, the monoclonal antibody will typically be combined with a pharmaceutically acceptable carrier. Such carriers include water, physiological saline, or oils.

The nucleic acid based antagonist of the present invention may be administered as a pharmaceutical formulation with or without suitable carriers (e.g., including, but not limited to, liposomes, nanoparticles, polymers, etc.) or be expressed from transcription units inserted into vectors. The vector may be a recombinant DNA or RNA vector, and includes DNA plasmids or viral vectors. The multi-target RNA molecule expressing viral vectors can be constructed based on, but not limited to, adeno-associated virus, retrovirus, adenovirus, or alphavirus.

Suitable routes of administration of a pharmaceutical composition of the nucleic acid based antagonist may, for example, include oral, rectal, transmucosal, or intestinal administration; parenteral delivery, including intramuscular, intravenous and subcutaneous injections.

Alternatively, the pharmaceutical composition of the nucleic acid based antagonist may be administered in a local rather than systemic manner, for example, via injection of the pharmaceutical composition directly into a target organ or cells, such as intramedullary, intrathecal, direct intraventricular, intraperitoneal, or intraocular injections, often in a depot or sustained release formulation. Intravesicular instillation, intranasal/inhalation delivery, and direct application to the skin or affected area are other possible means of local administration as is.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Except insofar as any conventional medium is incompatible with the active ingredient and its intended use, its use in any compositions is contemplated.

The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use.

### ABBREVIATIONS

Abs antibodies, polyclonal or monoclonal
aV integrin subunit alpha V
b3 integrin subunit beta 3
bFGF basic fibroblast growth factor
IFN interferon
Ig immunoglobulin
IgG immunoglobulin G
IL interleukin
MMP-1 matrix metalloproteinase 1
EMMPRIN extracellular matrix metalloproteinase inducer
EMMPRINR receptor
sEMMPRINR soluble EMMPRIN receptor
Mab monoclonal antibody
VEGF vascular endothelial growth factor
MMP matrix metalloproteinase

While having described the invention in general terms, the embodiments of the invention will be further disclosed in the following examples.

### EXAMPLE 1

### Recombinant EMMPRIN Stimulates MMP-1 Production by Human Microvascular Endothelial Cells from the Lung (HMVEC-L)

The effect of EMMPRIN on endothelial cells was investigated using microvascular endothelial cells, cells that are directly involved in angiogenesis process in vivo.

HMVEC-L cells were obtained from Clonetics, Walkersville, Md. (Cat# CC-2527, Lot# 8F1528). HMVEC-L cells were cultured under conditions recommended by the supplier. Briefly, cells were cultured in Endothelium Cell Growth Medium MV (EGM-2 MV, Clonetics, Cat#CC-3202) containing human epithelial growth factor (hEGF), hydrocortisone, human basic fibroblast growth factor (hFGF-B), vascular endothelial growth factor (VEGF), human insulin-like growth factor-1 (hIGF-1), ascorbic acid, gentamicin, 5% FBS, at 37° C., 5% CO₂.

Early passage cells (less than passage 3) were trypsinized and washed with RPMI-1640 once. Cells were resuspended in Dilution Medium (DM—Fibroblast Basic Medium+2% FBS) at a concentration of 5×100,000 cells/ml. 100 μl of the cell suspension containing 50,000 cells was added into each well in a 96 well cell culture plate. These wells were preloaded with soluble recombinant human EMMPRIN with final concentrations of 20 μg/ml, 6.67 μg/ml, 2.22 μg/ml, 0.74 μg/ml, 0.25 μg/ml, 0.08 μg/ml, and 0 μg/ml. Cells were incubated at 37° C., 5% CO₂, in a humidified incubator for 1 day and 3 days. Conditioned medium was collected from each well and subjected to MMP-1 activity assay.

Quantitative detection of MMP-1 activity in the conditioned medium was carried out using Human MMP-1 Activity Kit (R&D Systems, Minneapolis, Minn.) (Cat#F1M00). Briefly, MMP-1 in 150 μl of standard or sample was captured by anti-MMP-1 antibodies immobilized at the bottom of each well. Captured MMP-1 was subsequently activated by 4-aminophenylmercuric acetate (APMA). MMP substrate added into each well was cleaved by active MMP-1 and the resulting fluorescence was determined using SpectraFluor Plus Plate Reader (TECAN, Zurich, Switzerland) (Cat# F129005, Ser# 94747) with the following parameters: excitation wavelength at 320 nm and emission wavelength at 405 nm.

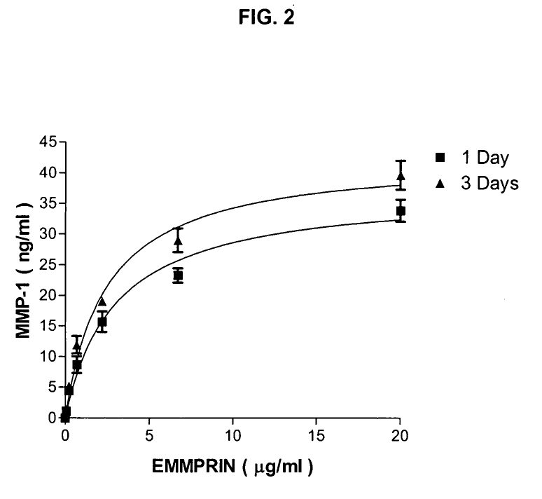

HMVEC-L cells were challenged with different concentrations of recombinant EMMPRIN to stimulate MMP-1 production. As shown in FIG. 2, EMMPRIN dose-dependently stimulated MMP-1 production in endothelial cells. HMVEC-L cells produced approximately 40 ng/ml MMP-1 when treated with 20 μg/ml EMMPRIN. This response of HMVEC-L to EMMPRIN stimulation was even stronger than that by NHLF cells, which produced only half of that amount of MMP-1 in response to the same treatment. The stimulation of MMP-1 production was first observed after one-day challenge and sustained for at least three days.

Results shown in FIG. 2, for the first time, demonstrated that EMMPRIN can directly stimulate MMP-1 expression by microvascular endothelial cells, the cells directly involved in angiogenesis, in a dose-dependent fashion.

### EXAMPLE 2

### Inhibition of EMMPRIN-Induced MMP-1 Production by an Anti-EMMPRIN Mab in HMVEC-L Cells

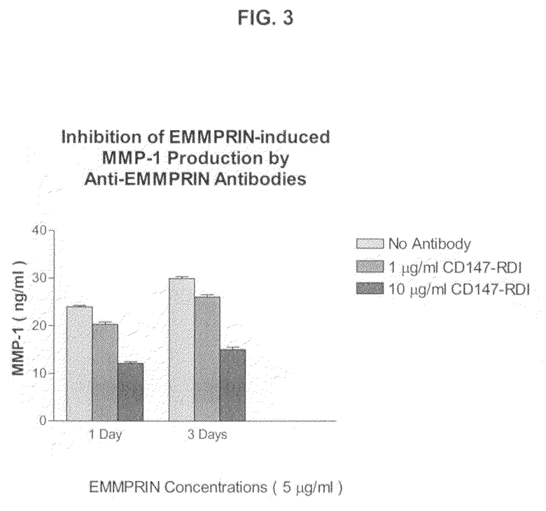

To further confirm the specificity of EMMPRIN-induced MMP-1 production, monoclonal antibodies against human EMMPRIN were included in the assay 15 minutes after cells were stimulated with EMMPRIN. At 10 μg/ml, the CD147-RDI/clone UM-8D6 (Research Diagnostics, Inc., Flanders, N.J.) significantly inhibited MMP-1 production by fibroblast cells induced by EMMPRIN (5 μg/ml) (FIG. 3). However, the other anti-EMMPRIN mAb (mouse anti-human CD147/EMMPRIN, clone HIM6, BD Pharmingen, San Diego, Calif.) was not able to inhibit MMP-1 production induced by EMMPRIN.

Our results demonstrated that the stimulation of MMP-1 production in HMVEC-L cells by EMMPRIN is specifically mediated by a unique epitope on EMMPRIN recognized by UM-8D6 but not HIM6.

### EXAMPLE 3

### Effects of EMMPRIN on Human Endothelial Cell Migration

The role of EMMPRIN in angiogenesis can also be directly investigated using in vitro cell migration and invasion assays. Human endothelial cells derived from primary tissue (umbilical cord) HUVEC cells were used in an in vitro system wherein endothelial cells are seeded in the top wells of the transwell system, in cell medium containing 1% FBS. In the bottom wells, culturing medium with 10% FBS will serve as a chemotactic source to induce cell migration or invasion. The top and bottom wells are separated by a membrane with pores of 8 μm in diameter. The membrane is either uncoated or coated with various extracellular matrix proteins, i.e., collagen, fibronectin, vitronectin, or Matrigel, for determining cell migration or invasion, respectively.

Materials and Methods MDA-MB-231 human breast cancer cells were purchased from ATCC (Manassas, Va.). Methods for transfection and establishment of MDA-MB-231 cells stably expressing different levels of EMMPRIN have been described previously (Tang, Y. et al. (2004) Mol. Cancer. Res. 2:73-80). The cells were transfected with the cDNA corresponding to human EMMPRIN open reading frame sense (MDA MB231 S1-3) or an antisense strand of the same ORF (MDA MB231 AS1-5 and MDA MB231 AS2-5) in pcDNA3.1 TOPO vector (Invitrogen, Carlsbad, Calif.). Cells transfected with the empty vector were used as a second control (Vector).

Endothelial cell (HUVEC) migration was evaluated using QCM™-Collagen I Quantitative cell migration assay kit (Chemicon, Temecula, Calif.). HUVEC cells (100,000 in 100 μl serum-free medium) were added to the top compartment. Serum-free media conditioned by MDA MB231 cells: WT, Vector, S1-3, AS1-5, or AS2-5 was used as the chemoattractant source in the bottom compartment of chamber. In a second experiment, anti-VEGF mAb (R&D Systems, Minneapolis, Minn.) was added into the bottom compartment at various concentrations to neutralize VEGF biological activity. Cell migration assays were carried out at 37° C. for 6 hours. Insert filters were fixed and cells remaining in the top compartment were removed. Filters were stained with Gentsian violet and the number of migrated cells determined using a microscopic imaging system (Pro-Plus 3D Imaging System).

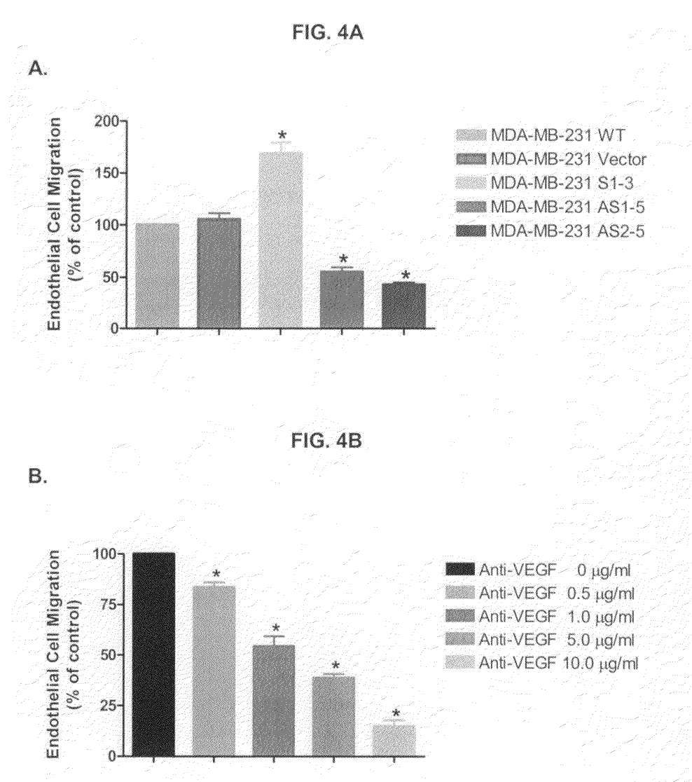

FIG. 4A shows the relative level of HUVEC cell migration induced by conditioned medium derived from the various MDA-MB-231 cell constructs. WT cell-induced migration was assigned 100%. Error bars represent standard deviations of triplicate data points. Significant differences by Students T-test (*) was at the p<0.01 value compared to endothelial cell migration induced by WT cells. FIG. 4B shows that a neutralizing antibodies to VEGF inhibited endothelial cell migration stimulated by serum-free medium conditioned by MDA-MB-231 EMMPRIN S1-3 tumor cells, assigned as 100%, in a dose-dependent manner. Error bars represent standard deviations of triplicate data points; *p<0.01 compared to endothelial cell migration in the absence of the anti-VEGF mAb.

These data demonstrate the involvement of VEGF in EMMPRIN-induced endothelial cell migration.

### EXAMPLE 4

### Effects of EMMPRIN on HMVEC-L Cell Tube Formation

The role of EMMPRIN in angiogenesis can be shown using in vitro tube formation assays. When seeded on Matrigel, HMVEC cells initiate a spontaneous differentiation process to form capillary-like tube structure. This in vitro differentiation mimics in vivo angiogenesis process and is often employed in angiogenesis studies.

It is predicted that EMMPRIN will change the properties of endothelial cells by stimulating MMP expression, and therefore stimulate cell migration and invasion. An enhanced tube formation will occur when these cells are stimulated with EMMPRIN.

The specificity of EMMPRIN in tube formation is investigated using monoclonal antibodies against human EMMPRIN.

### EXAMPLE 5

### Effects of EMMPRIN on angiogenesis in Vivo cl Matrigel Plug Assay

The role of EMMPRIN in angiogenesis is directly investigated in vivo using Matrigel plug assays. Matrigel is a solubilized basement membrane preparation extracted from the Engel-Holm-Swarm (EHS) mouse sarcoma, a tumor rich in extracellular matrix proteins. The major component is laminin, but Matrigel also contains trace amounts of fibroblast growth factor, TGF-beta, tissue plasminogen activator, and other growth factors that occur naturally in the EHS tumor. Matrigel is the basis for several types of tumor cell invasion assays and provides the necessary substrate for the study of angiogenesis. Matrigel forms a soft gel plug when injected subcutaneously into mice or rats and supports an intense vascular response when supplemented with angiogenic factors.

Matrigel plugs containing suboptimal doses of angiogenic growth factors, such as basic fibroblast growth factor (FGF), or vascular endothelial cell growth factor (VEGF) can be implanted into mice to induce angiogenesis in vivo. Some of these plugs are supplemented with various doses of recombinant EMMPRIN. Since EMMPRIN induces endothelial cell migration and MMP production by endothelial cells, an increase in angiogenesis due to enhanced cell migration and invasion through Matrigel is expected.

These effects of EMMPRIN as tested in the Matrigel plug angiogenesis assay can be used to demonstrate the activity of EMMPRIN antagonists, such as siRNA, shRNA, DNAzymes, or anti-EMMPRIN antibodies, in preventing angiogenesis.

### EXAMPLE 6

### Effects of EMMPRIN on Angiogenesis in Vivo

### Corneal Pocket Assay

Similarly, the role of EMMPRIN in angiogenesis is directly investigated in vivo using corneal pocket assays.

Polymer discs containing angiogenic growth factors, such as basic fibroblast growth factor (FGF), or vascular endothelial cell growth factor (VEGF), are implanted into a corneal pocket in order to evoke vascular outgrowth from the peripherally located limbal vasculature. A combination of suboptimal doses of angiogenic growth factors supplemented with various doses of recombinant EMMPRIN is used. Since EMMPRIN will induce MMP production by endothelial cells, an increase in angiogenesis due to enhanced endothelial cell migration and invasion is expected.

The specificity of EMMPRIN in corneal pocket angiogenesis assay is investigated using EMMPRIN antagonists, such as siRNA, shRNA, DNAzymes, or anti-EMMPRIN antibodies.

### EXAMPLE 7

### Effects of EMMPRIN on Angiogenesis

### Stimulation of VEGF Production and Release Mediated by MMP

It has been reported that EMMPRIN also stimulates the expression of membrane-type matrix metalloproteinase 1 (MT1-MMP) [Sameshima et al. 2000b]. MT1-MMP in turns stimulates expression of VEGF, one of the most potent angiogenic growth factors, resulting in enhanced angiogenesis [Deryugina et al. 2002; Sounni et al. 2002]. However, the direct link between EMMPRIN and VEGF expression, and angiogenesis has yet to be established.

Using either recombinant EMMPRIN or tumor cells expressing altered levels of EMMPRIN, the link between EMMPRIN and VEGF, in both in vitro and in vivo settings can be demonstrated. As an increase in the VEGF level promotes angiogenesis, in addition to EMMPRIN-induced endothelial cell migration and invasion, the resulting effect on tumor invasiveness and growth rate is made evident.

Materials and Methods MDA-MB-231 human breast cancer cells were purchased from ATCC (Manassas, Va.). Methods for transfection and establishment of MDA-MB-231 cells stably expressing different levels of EMMPRIN have been described previously (Tang, Y. et al. (2004) Mol. Cancer. Res. 2:73-80). The cells were transfected with the cDNA corresponding to human EMMPRIN open reading frame sense (MDA MB231 S1-3) or an antisense strand of the same ORF (MDA MB231 AS1-5 and MDA MB231 AS2-5) in pcDNA3.1 TOPO vector (Invitrogen, Carlsbad, Calif.).

Normal human lung or dermal fibroblast cells (NHLF or NHDF), and human microvascular endothelial cells from the lung (HMVEC-L) or human umbilical vein endothelial cells (HUVEC) were obtained from (Clonetics, Walkersville, Md.) and were cultured in Fibroblast Growth Medium or Endothelial Growth Medium-2 (EGM-2) respectively.

For co-culture studies of cancer and fibroblast cells, 100,000 cancer cells (MDI MB231 WT, S1-3, AS1-5, or AS 2-5) were cultured together with 200,000 NHDF cells in a six-well culture plate in complete DMEM. After 24 hours, the medium was replaced with serum-free DMEM and the cultures continued for 2 days. The medium was replaced with fresh serum free DMEM and the cultures maintained for an additional 3 days at which time the medium was collected and analyzed. The cells were lysed with Tris-buffered saline plus 1% NP40 to determine cell-associated EMMPRIN.

The relative amount of EMMPRIN expressed in 10 ug of total cell protein was determined by Western blot analysis using scanning densitometry, by quantitative ELISA using anti-EMMPRIN antibody (RDI-147, Research diagnostics) as described (Tang et al. 2004) and on the cell surface by fluorescence activated cell analysis (FAC analysis). The FAC analysis confirmed that cell surface EMMPRIN was absent on cells transfected with antisense constructs (data not shown). The presence of MMP-2 and MMP-9 in serum-free medium or tumor extracts was determined by substrate SDS-PAGE zymography using 10 ug of total protein. Proteolytic activities on the gel were detected as clear bands on a blue background of undigested and stained gelatin. ELISA measurements of human or mouse MMP-2, MMP-9 and VEGF concentrations were performed using Quantikine ELISA kits from R&D Systems, according to the manufacturer's instructions. Each sample was analyzed in triplicates. Briefly, MMP-2, MMP-9 or VEGF contained in 100 μl of standard or samples (equivalent of 50 μg of total protein) were captured by anti-MMP-2-, anti-MMP-9-, or anti-VEGF-antibodies immobilized on the bottom of assay wells. After washing, the MMP or VEGF specific antibody was used to quantitate the amount present.

Results The transfected cells had altered levels of total EMMPRIN when grown in cell culture conditions (Table 1). S1-3 cells had approximately twice the level of WT cells and 4-fold that of the AS cells.

TABLE 1

| | EMMPRIN Expression | | VEGF |
|---|---|---|---|
| MDA MB231 cell | (Rel. Amount) | MMP Detected | (pg/ml) |
| WT | 100% | None | 208.1 |
| Vector | ND | | 175.5 |
| S1-3 | 190% | MMP-2 (weak) | 310.1 |
| AS1-5 | 47% | | 64.6 |
| AS2-5 | 62% | | 108.7 |

TABLE 2

| MDA MB231 cell | MMP Detected | VEGF (pg/ml) |
|---|---|---|
| WT | MMP-2 | 306.3 |
| | MMP-9 | |
| WT + 1.10 PA | ND | 240 |
| WT + anti-CD147 | ND | 220 |
| None (NHDF | MMP-2 | 19.5 |
| only) | | |
| S1-3 | MMP-2 | 416.1 |
| | MMP-9 | |
| AS1-5 | None | 134.7 |
| AS2-5 | None | 154.3 |

These data show a relationship between tumor cell expression of EMMPRIN, MMP expression, and VEGF levels in the engineered tumor cells alone. The co-culture data (TABLE 2) show a supra-additive amount of VEGF is produced when NHDF are present with either WT human breast tumor cells or those overexpressing EMMPRIN (S1-3).

### EXAMPLE 8

### Stimulation of in Vivo Tumor Angiogenesis by EMMPRIN

The stimulatory effects of tumor cell derived EMMPRIN on angiogenesis, the formation of new blood vessels, was directly assessed in vivo. Human breast cancer cells, MDA MB 231, were engineered to express different levels of EMMPRIN protein using recombinant DNA technology. Sense EMMPRIN cells were created that represent a cell population derived from a single cell clone that was stably transfected with a mammalian expression vector encoding the full-length human EMMPRIN. Antisense cells were generated by transfecting MDA MB 231 cells with a mammalian expression vector encoding the full-length human EMMPRIN in the antisense orientation. Sense cells constitutively express increased levels of EMMPRIN, and antisense cells express decreased levels of EMMPRIN due to inhibition of protein translation by the antisense RNA (See Example 7). These cells, together with wild-type cells, were implanted subcutaneously into nude mice. Tumor angiogenesis was assessed in tumors derived from these cells.

All procedures involving animals and their care were conducted in conformity with the company ICAUC guidelines that are in compliance with the NIH standard. Four-week-old female CD1 Nu/Nu mice were obtained from Charles River Laboratories, and acclimated for 10-14 days prior to the experiment.

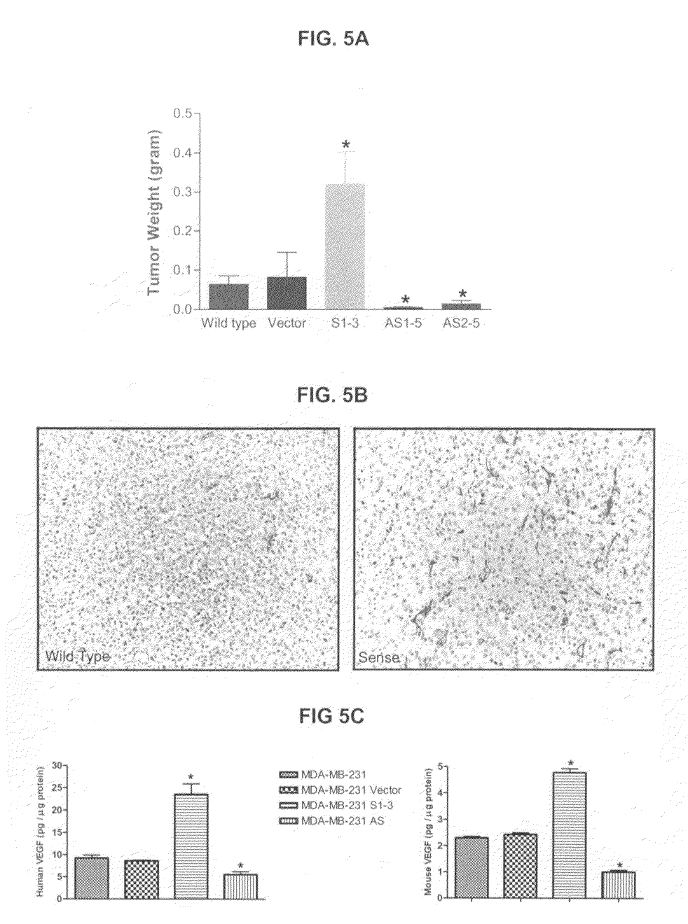

Comparing with tumors derived from wild type or vector control tumor cells, a 5-fold increase in final tumor weight was seen in S1-3 tumors produced by the EMMPRIN-overexpressing cells (FIG. 5A). AS1-5 and AS2-5 cells produced significantly smaller size (p=0.0242 and 0.0439, respectively) compared to the unaltered control cells, WT, or those transfected with empty vector, Vector, during the same period of time (FIG. 5A).

As shown in FIG. 5B, increased angiogenesis was evidenced by numerous new capillary blood vessels in tumors derived from sense cells, but not in tumors derived from wild-type and antisense cells.

Human VEGF level was 2.6-fold higher in xenografts tissue produced by S1-3 cells (23.53 pg/ug total protein) as compared to WT (9.24) and Vector control cells (8.60 pg/ug total protein (p=0.0043 sense vs wild type) (FIG. 5C). Tumor tissue from AS cells, with suppressed EMMPRIN levels had 40.4% less human VEGF or 5.5 pg/ug total protein (p=0.0177 compared WT).

More importantly, the impact of increased EMMPRIN level on tumor cell surface to VEGF expression in tumor tissues extended beyond tumor cells. Concomitant with stimulation of tumor VEGF production, mouse stromal VEGF production also increased in mice with S1-3 tumors. Levels of host-derived VEGF escalated 2.1-fold from 23 and 24 pg/mg total protein in wild type and vector control tumors to 48 pg/ug total protein in sense tumors (p=0.00009 sense vs WT) (FIG. 5C). Mouse tissue VEGF from mice given AS cells had less VEGF by −56.6% or 10 pg/mg total protein (p=0.00013 compared with WT).

Thus, both tumor and host cell-derived VEGF levels followed EMMPRIN-levels in EMMPRIN-modified cell derived tumors and these trends. These observations support a new paradigm in which tumor EMMPRIN mediates an active interaction between tumor and stromal compartments to stimulate VEGF production and subsequently tumor angiogenesis and growth in vivo.

### EXAMPLE 9

### Effect of Tumor EMMPRIN on Tumor Tissue Environment with Respect to MMPs and VEGF

Human breast tumor cells, as described in Example 3, were used to assess the effect of increased of decreased EMMPRIN on tumor tissue and tumor stroma (fibroblasts, endothelial cells, and other ancillary cells) in vivo.

On day 0, at approximately 6 weeks of age, mice were assigned to each of 5 groups consisting of 8 mice per group. Animals were inoculated with 107 cells in 0.1 mL of cell suspension subcutaneously in the right flank region. Tumor growth was monitored weekly by caliper measurement and tumor volume (mm³) were calculated based on the formula [length×width×width]/2. At termination of the experiment, all animals were euthanized via CO₂ asphyxiation. Primary tumors were excised, weighed, rinsed in ice-cold PBS and processed for histological/microscopic examination. Tissue specimens and sections were also snap-frozen in liquid nitrogen for protein extraction and biochemical analysis.

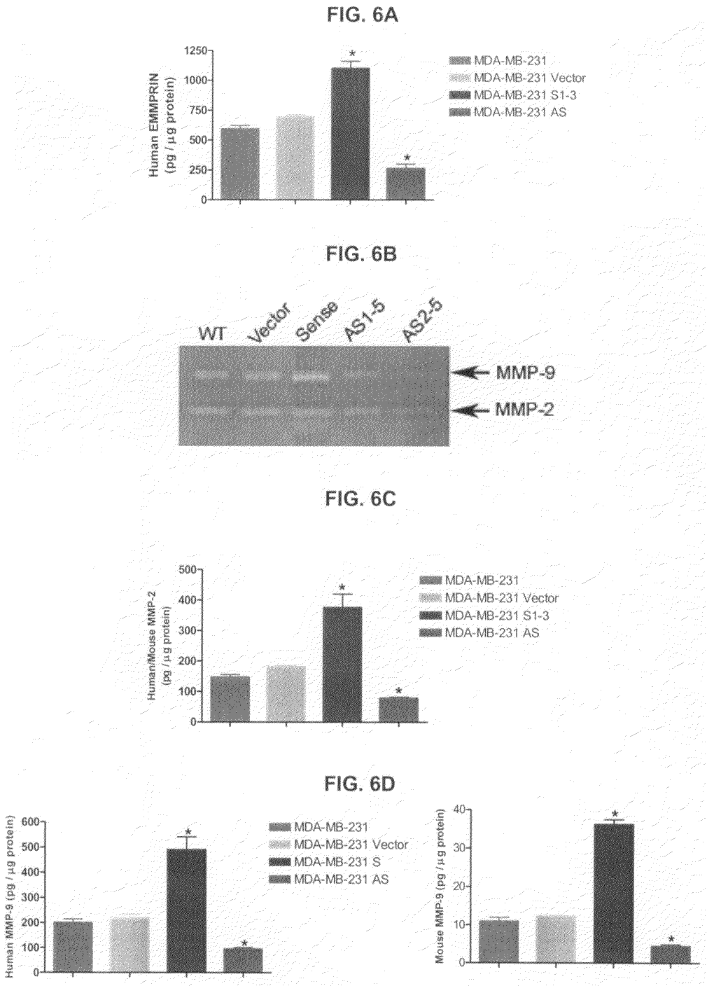

Human EMMPRIN levels were quantitatively assessed with ELISA analysis, which demonstrated a considerable gain of EMMPRIN level in sense tumors (109.8 pg/ug of total protein), and conversely a greatly suppressed level in antisense tumors (26.0 pg/ug of total protein), compared with 59.0 pg/ug of total protein in wild type tumors (p=0.000048 and 0.000077 for sense and antisense tumors vs wild type respectively) (FIG. 6A). This stable effect of EMMPRIN expression on transfected tumor cells was subsequently translated into influences on MMP expression in vivo. As expected, substrate zymography analysis of tumor tissue extracts revealed increased levels of MMP-2 and MMP-9 activities in EMMPRIN sense tumors, and lowered levels when EMMPRIN expression was suppressed (FIG. 6B). MMP levels in both tumor and host compartments were then quantitatively determined by biochemical analysis. When EMMPRIN was over-expressed in tumor cells, both human MMP-2 and human MMP-9 expression levels in the resulting xenograft tumors were elevated by approximately 2.5-fold (p=0.0068 and 0.0056 compared with wild type tumors respectively) (FIG. 6C). Conversely, a 2-fold decrease in the expression of these two MMPs was observed when EMMPRIN was inhibited in antisense tumors (p=0.0026 and 0.0035 compared with wild type tumors respectively) (FIG. 6C). The effect of tumor EMMPRIN expression on host MMP-9 activity associated with stromal cells was even greater than that on tumor MMPs.

Thus, the change in tumor cell surface EMMPRIN was capable of inducing a 3.3-fold increase or a 59.3% decrease in mouse MMP-9 expression in sense or antisense tumor nodules, respectively (p=0.00013 and 0.0047 compared with wild type tumors) (FIG. 6D).

Visualization of Tumor EMMPRIN-MMP Systems In Vivo

Tumor

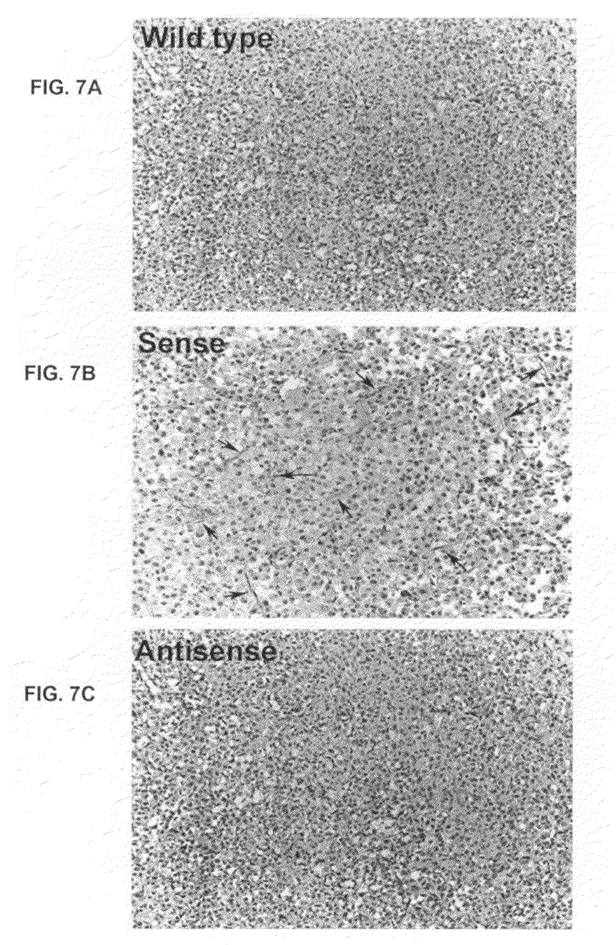

The difference in angiogenic activity is between tumors produced by the cells overexpressing EMMPRIN and the WT or under expressing cells, AS, was clearly visible (FIG. 7).

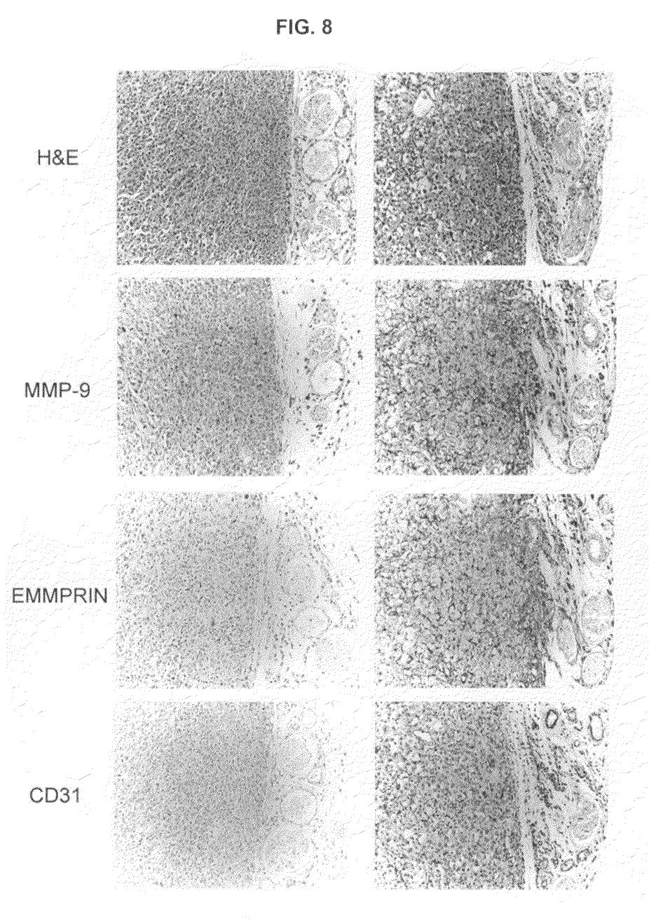

The effect of tumor EMMPRIN expression on host EMMPRIN-MMP system was further studied in immunohistochemical analysis of the xenograft tumors. In tumors produced by tumor cells that over-express EMMPRIN (MDA MB231 S1-3), up-regulation of both mouse MMP-9 and EMMPRIN was detected in stromal cells. The expression of these two proteins was restricted to mouse cells and was not detected in xenograft human tumor cells (FIG. 8). Interestingly, in addition to the staining found in capsules surrounding the tumor or in fibroblast cells in the stromal compartments infiltrated into the tumor tissues, both mouse EMMPRIN and MMP-9 were highly up-regulated around blood vessel-like structures (FIG. 8). Co-localization of MMP-9 and EMMPRIN around angiogenic blood vessels was further supported by overlapping distribution of mouse MMP-9, EMMPRIN and that of CD31, a blood vessel marker (FIG. 8). In contrast, there were only minimal levels of MMP-9 and EMMPRIN expression in tumors produced by Vector control tumor cells. In these tumors, MMP-9 was mainly detected in macrophage-like cells, and EMMPRIN was detected at very low levels in some fibroblast cells (FIG. 8).

### EXAMPLE 10

### Production and Characterization of Anti-Angiogenic Anti-EMMPRIN Monoclonal Antibody

Anti-angiogenic anti-EMMPRIN antibodies can be prepared using standard procedures and screened using the properties described herein for anti-angiogenic EMMPRIN antagonists.

Materials and Methods: Three 12-14 week old Balb/c mice were obtained from Charles River Laboratories. Two mice each received combination intradermal and intraperitoneal injections of 25 pg rHuEMMPRIN (R&D Systems) (12.5 μg/site) in 75 μL PBS emulsified in an equal amount of Freund's complete adjuvant on day 0, and 25 μg rHuEMMPRIN in 75 μL PBS emulsified in an equal amount of Freund's incomplete adjuvant on days 14, 28 and 51. The third mouse received an initial injection of 25 μg of rHuEMMPRIN+0.33×10⁵ U murine IFNα+0.33×10⁵ U murine IFNβ (Biosource) in 100 μl PBS administered S.Q. at the base of the tail. On days 2 and 3, the mouse received additional injections of 0.33×10⁵ U IFNα+0.33×10⁵ U IFNβ in 100 μL PBS administered S.Q. at the base of the tail. Several weeks later, the mouse was boosted with 25 μg EMMPRIN+100 pg anti-murine CD40 agonist Mab (R&D Systems) administered S.Q. at the base of the tail.

The mice were bled at various time-points throughout the immunization schedule. Blood collections were performed by retro-orbital puncture and serum was collected for titer determination by solid phase EIA. Once titer plateau was obtained, the mice received their final booster of 25 pg of EMMPRIN in PBS given intravenously (IV). Three days later the mice were euthanized by CO₂ asphyxiation, and the spleens were aseptically removed and immersed in 10 mL cold PBS containing 100 U/mL penicillin, 100 μg/mL streptomycin, and 0.25 pg/mL amphotericin B (PBS/PSA). Lymphocytes were harvested by sterilely passing cells though a wire mesh screen immersed in cold PBS/PSA. The cells were washed once in cold PSA/PBS, counted using Trypan blue dye exclusion and resuspended in 10 mL PBS.

Characterization of Anti-Human EMMPRIN Antibodies Enzyme immunoassays (EIAs) were used to test hybridoma cell supernatants for the presence of human anti-EMMPRIN Mabs. Briefly, plates (Nunc-Maxisorp) were coated overnight with human EMMPRIN at 1 pg/mL in PBS. After washing in 0.15 M saline containing 0.02% (w/v) Tween 20, the wells were blocked with 1% (w/v) bovine serum albumin (BSA) in PBS for 1 hr at 37° C. Undiluted hybridoma supernatants were incubated on coated plates for 1 hour at 37° C. The plates were washed and then incubated with HRP-labeled goat anti-murine IgG, Fc specific (Sigma) diluted 1:10,000 in 1% BSA/PBS for 30 minutes at 37° C. Plates were again washed then incubated for 15 minutes at RT with 100 μL/well of citrate-phosphate substrate solution (0.1 M citric acid and 0.2 M sodium phosphate, 0.01% H₂O₂, and 1 mg/mL o-phenylenediamine dihydrochloride). Substrate development was stopped by the addition of 4N sulfuric acid at 25 μl/well and the absorbance was measured at 490 nm via an automated plate spectrophotometer. All reactive hybrid cell lines were subcloned twice by limiting dilution at 1 cell/well in cloning plates. The homogeneous cell lines were cryopreserved in freezing medium (90% FBS, 10% DMSO) and stored in liquid nitrogen.

To identify the isotype of the murine anti-human EMMPRIN antibodies, the Monoclonal Antibody Isotyping Kit-IsoStrip, Dipstick Format (Roche) was used as per the manufacturer instructions. Briefly, culture supernatant was diluted 1:10 in PBS and added to the development tube. The dipstick was added to the development tube and incubated at RT for approximately ten minutes. Isotypes were determined by visual assessment following incubation. A list of eighteen different hybridoma clones that secrete a murine IgG Mab that specifically binds to human EMMPRIN is shown in Table 3.

The biologic activity of recombinant EMMPRIN used as antigen protein was assayed by its ability to stimulate production of MMP-1 from EMMPRIN stimulated in fibroblast cells was performed as described (Guo, Zucker, Gordon, Toole and Biswas, (1997), J Biol Chem 272: 24-7)(24)), modified by using highly homogenous primary human fibroblast cells of less than three passages and modified stimulation conditions. Only highly pure fibroblast cells that were confirmed being negative for cytokeratin 18, cytokeratin 19, factor VII-related antigen, and alpha actin were used in the assay. The magnitude of response to EMMPRIN stimulation was dependent on the passage of fibroblast cells. Cells of earlier passages responded more potently in producing increased amounts of MMP, compared to cells that have been cultured for more than three passages. In addition, a new cell challenge method was used. Instead of adding recombinant EMMPRIN to adherent cells, recombinant EMMPRIN was preloaded in testing wells. Suspended cells were then added into these wells and were directly exposed to recombinant EMMPRIN. This new challenge procedure ensures the maximal exposure of cell surface receptors, which are likely expressed on the basolateral surfaces and could be out of access in adherent cells, to EMMPRIN for optimal assay sensitivity.

Recombinant EMMPRIN corresponding to the extracellular domain of human EMMPRIN protein was produced in NSO cells (R&D Systems, Minneapolis, Minn.). MMP-1 activity in serum-free medium conditioned by fibroblast cells treated with different amounts of recombinant EMMPRIN protein was quantitatively determined using an MMP-1 Activity Assay Kit according to product manual (R&D Systems, Minneapolis, Minn.). Briefly, MMP-1 contained in 150 μl of standards or samples was captured by anti-MMP-1 antibodies immobilized on the bottom of assay wells. Captured MMP-1 was subsequently activated by 4-aminophenylmercuric acetate (APMA). MMP substrate added into each well was cleaved by activated MMP-1 and the resulting fluorescence was determined using SpectraFluor Plus Plate Reader (TECAN, Research Triangle Park, N.C.) with the following parameters: excitation wavelength at 320 nm and emission wavelength at 405 nm. To determine the inhibitory activity of anti-EMMPRIN antibodies, antibodies were added into cell culture after cells were stimulated with recombinant EMMPRIN for 15 minutes.

The panel of monoclonal antibodies was all screened for these two activities in addition to Isotyping (TABLE 3).

TABLE 3

| NO# | Isotype | MMP-1 | Co-culture |
|---|---|---|---|
| 1 | IgG2bk | N | P |
| 2 | IgG1k | N | N |
| 3 | IgG1k | N | N |
| 4 | IgG1k | N | N |
| 5 | IgG1k | N/A | N/A |
| 6 | IgG2bk | N | N |
| 7 | IgG1k | P | P |
| 8 | IgG1k | N | P |
| 9 | IgG1k | N | N |
| 10 | IgG2bk | N | N/A |
| 11 | IgG1k | N | N |
| 12 | IgG1k | N* | N |
| 13 | IgG1k | N | N/A |
| 14 | IgG1k | N | N/A |
| 15 | IgG1k | N | N/A |
| 16 | IgG1k | N | N/A |
| 17 | IgG1k | N | N/A |
| 18 | IgG1k | N | N/A |
| N/A (Anti- | IgG1k | P | P |
| CD147) | | | |

P = positive; N = negative

The antibody designated NO 7 met the initial selection criteria for an anti-angiogenic anti-EMMPRIN Mab.

Inhibition of MMP-2 Production in Co-Culture of Tumor Cells and Fibroblast Cells

The co-culture assay was performed as previously described above using normal human dermal fibroblasts and human melanoma tumor cells (G361) were used and either the commercial antibody RDI CD147 or NO 7 were added to the cultures. Three days after the last change of serum free medium, the amount of MMP-2 was quantitated. The data showed that NO 7 was capable of inhibiting MMP-2 production in these co-cultures as did the commercial antibody.

### EXAMPLE 11

### SiRNA and DNAzyme Antagonists

The role of EMMPRIN in tumour progression can be elucidated by modulating gene expression by the RNAi pathway or by the use of specific DNAzymes. Similarly, EMMPRIN can be overexpressed by the use of vectors containing a strong promoter. Stable cell lines suppressed for EMMPRIN expression and overexpressing EMMPRIN were generated. These cell lines can then be used in in vivo xenograft and in vitro functional studies allowing the rapid validation of EMMPRIN as a cancer target. To this end, siRNAs specific for EMMPRIN were screened and then effective siRNAs were cloned into vectors as hairpins allowing the generation of stable cell lines. Thirty-five EMMPRIN-specific DNAzymes were also designed and tested. Overexpression clones were also generated.

Materials and Methods

Cell Lines

MDA MB 231 and MDA MB 435S-GFP cells were maintained in tissue culture in DMEM supplemented with 10% FBS, 2 mM L-glu and 10 U/ml penicillin/streptomycin (Gibco).

SiRNA Design

Candidate siRNAs were designed to the cDNA sequence of EMMPRIN (accession number NM_{—}001728) with reference to the Ambion (www.ambion.com) and Dharmacon (www.dharmacon.com) siRNA design centres and using the Tuschl rules. The resulting candidates were blasted against the human genome to avoid complementarity with multiple targets and then six candidates that were evenly spaced along the cDNA, including the 3′ and 5′ untranslated regions, were chosen for synthesis (Proligo).

Transient siRNA Transfection

Cells (2×10⁵) were seeded into 6 well dishes and transfected with 60 nM of siRNA 48 hours later using Lipofectamine 2000 (Invitrogen), according to the manufacturer's instructions. EMMPRIN expression was measured at 24 and 48 hours post-transfection by antibody staining and flow cytometry.

pSilencer Vectors

DNA hairpin oligos corresponding to the sequence of the siRNAs were synthesized using Ambion's shRNA design centre and their default loop sequence. Oligos were cloned into Ambion's pSilencer 2.1 and 3.1 vectors according to the manufacturer's instructions. Large-scale preparations (HiSpeed Plasmid Maxi Kit, Qiagen) of the vectors were made and sequenced to confirm the presence of the correct shRNA sequence. Large-scale preparations of the 2.1 and 3.1 negative control vectors were also made. pSilencer vectors were linearized with the AfIIII restriction endonuclease prior to transfection.

Generation of Stable Clones

Cells (3×10⁶) were seeded into 100 mm² dishes and transfected with vector DNA (30 μg) on the following day, either by electroporation or using Lipofectamine 2000 (Invitrogen), according to the manufacturer's instructions. On the following day, cells were harvested using trypsin (Gibco) and re-seeded into 100 mm² dishes at 10- and 100-fold dilutions in DMEM containing hygromycin (250 pg/ml) or puromycin (200 ng/ml). Clones were grown under antibiotic selection, expanded to 6-well dishes and then screened for EMMPRIN expression by antibody staining and flow cytometry.

Antibody Staining and Flow Cytometry

Cells were harvested and washed in PBS containing 5% (v/v) FBS (blocking buffer). Cells were then stained directly with a FITC-conjugated mouse anti-EMMPRIN antibody (1:10 dilution; all antibodies from BD PharMingen) or indirectly with a purified mouse anti-EMMPRIN antibody (1:50 dilution), washed with blocking buffer and followed by a R-PE-conjugated rat anti-mouse IgG1 secondary antibody (1:50 dilution). Isotype control antibodies were used as negative controls. Antibodies were diluted in blocking buffer. After washing in blocking buffer, cells were resuspended in PBS and EMMPRIN-positive cells were analyzed by flow cytometry. Results were expressed as the percentage of EMMPRIN expression on clones compared to untreated parental cells.

Western Analysis

Thawed cell pellets were lysed in RIPA buffer containing protease inhibitors for 30 minutes on ice. Lysates were centrifuged at 12,000 rpm for 15 minutes at 4° C. and supernatants were adjusted to 25 μg of total protein (quantified by DC protein assay, BioRad) per sample in Laemmli sample buffer. Samples were incubated at 70° C. for 10 min and proteins were fractionated on 10% sodium dodecyl sulphate-polyacrylamide gels (Invitrogen). After electrophoresis, gels were blotted onto polyvinylidine fluoride membranes. Membranes were blocked with 5% skimmed milk powder (w/v) in TBS-T (TBS containing Tween 20 (0.2% v/v) at room temperature for 1 hour, then washed and incubated overnight in TBS-T. Membranes were then probed with the purified mouse anti-EMMPRIN antibody (1:2000 dilution) in blocking buffer for 1 hour at room temperature, washed with TBS-T and probed with a goat anti-mouse IgG horseradish peroxidase-conjugated secondary antibody (1:2000 dilution; Santa Cruz Biotechnology) in blocking buffer for 1 hour at room temperature, washed with TBS-T, followed by incubation with enhanced chemiluminescence (Amersham Pharmacia Biotech) for 1 min. Membranes were exposed to radiography film and then developed. Membranes were then washed again in TBS-T and re-probed and developed as before, but using an anti-β-actin primary antibody (1:5000 dilution; Sigma) as an internal control.

Northern Analysis

RNA was extracted from thawed cell pellets by resuspending in Trizol, adding chloroform and centrifuging at 14,000 rpm for 15 minutes at 4° C. {Sambrook, 1989}. RNA was precipitated from the aqueous phase by the addition of isopropanol and incubation at room temperature for 10 min. Pellets were washed with 75% ethanol and resuspended in DEPC-treated water by incubation at 55° C. for 10 min. RNA was then fractionated by electrophoresis in 0.8% agarose gels in water-buffer-formaldehyde mixture {Sambrook, 1989}. Total RNA (20 μg) was loaded in each lane after denaturation in formaldehyde-formamide-buffer mixture at 65° C., 15 min, in the presence of EtBr, followed by 5 min on ice. After electrophoresis, the RNA was transferred onto Hybond N+ filters (Amersham Biosciences) in a 20×SSC solution, as described in the manufacturer's manual. An EMMPRIN PCR product (untagged) was radioactively-labelled by random primer incorporation [γ-³²P]dCTP {Sambrook, 1989}, purified in a BioSpin chromatography column (BioRad) and used as a probe. Hybridisation of radioactive probes (50 ng) to RNA immobilized on Hybond N+filters was performed in ExpressHyb hybridisation solution (BD Biosciences) according to the manufacturer's instructions. After phosphoimaging, the membrane was stripped in boiling water containing 0.1% SDS and re-probed with an 18S probe that was prepared by end-labelling with [Y-³²P]ATP {Sambrook, 1989}, as an internal control. Quantification of transcript levels was carried out in Image Quant using the local average and normalized to the levels of 18S mRNA in each sample.

Quantitative RT-PCR (Qzyme)

Qzyme analysis {Todd, 2000} was performed on samples using the primer pair below at a 3′ to 5′ primer ratio of 10:1 and a magnesium concentration of 2.8 mM. Reactions were run on a Corbett Rotorgene and the thermal cycling parameters were 1×50° C. for 30 min, 1×95° C. for 10 min, 10×(95° C. for 15 s, 65° C. (−1 C/cycle) for 1 min, 50×(95° C. for 15 s, 55° C. for 1 min).

5′ primer:

(SEQ ID NO:1)

5′ CAGCACAACCTCGTTGTAGCTAGCCTCACCAACCGTGAAGTCGTCAG
AACACATC 3′
3′ primer:

(SEQ ID NO:2)

5′ AGAGTCAGTGATCTTGTACC 3′

The levels of EMMPRIN transcript in unknown samples (250 ng of total RNA) were measured relative to the levels of EMMPRIN transcript in dilutions of RNA from untransfected MDA MB 231 cells (standard curve constructed using 500 ng total RNA, and then seven 5-fold dilutions). Standards were run in duplicate, unknowns in triplicate and no template controls (NTC) in quadruplicate. EMMPRIN transcript levels were normalized by comparison to HPRT levels in the same RNA samples.

The amount of EMMPRIN transcript in the samples was expressed as a normalized ng equivalent of RNA from untransfected MDA MB 231 cells as follows:

Ng equivalent=(mean sample amount/mean HPRT amount)×250

The standard deviation in the ng equivalent was calculated as follows:

$$ST DEV = \sqrt{( {( \frac{SDEMMsample}{meanEMMsample} )}^{2} + {( \frac{SDHPRT}{meanHPRT} )}^{2} )} \times ngequiv$$

Optimization of DNAzyme Transfection

MDA MB 231 cells were transfected in 6-well plates (3×10⁵) and 60 mm dishes (5×10⁵) with a FAM-labelled DNAzyme using varying amounts of DNAzyme and lipofectamine. FAM positive events were analyzed by flow cytometry at 24 hours post-transfection.

Screening of EMMPRIN-Specific DNAzymes

A first set of twenty DNAzymes targeting EMMPRIN cDNA (accession no. NM_{—}001728) were designed according to accepted protocols. In brief, 19-mer sequences within the cDNA were selected with an A at position 9 and a C or T at position 10. The corresponding DNAzymes for these sites were then designed by replacing the central C or T with the 10-23 catalytic core motif {Santoro, 1997;Santoro, 1998}. Candidates were then examined with respect to free energy and 90 suitable candidates remained. These 90 were blasted against the human genome and aligned with the EMMPRIN mRNA sequence. Twenty DNAzymes that were roughly evenly spaced along the EMMPRIN mRNA sequence were selected for synthesis (GeneWorks) and each one contained four phosphorothioate linkages (terminal two residues per arm). DNAzymes corresponding to the positions of all six of the EMMPRIN-specific siRNAs were included. Prior to transfection, DNAzymes were end-labelled with [γ-³²P]ATP {Sambrook, 1989}, run on a 16% sodium dodecyl sulphate-polyacrylamide gel and the DNAzymes were visualized by phosphoimaging for quality control purposes.

An additional set of 15 DNAzymes was selected according to a different approach. In this approach, the target mRNA was folded in silico using OligoWalk software; this software predicts the secondary structure of a target mRNA under physiological conditions in a similar fashion to the Mfold software. Areas predicted to be largely free of secondary structure and which featured AU or GU sites within the mRNA sequence were selected. The DNAzymes matching these sites and having 9+9 nt arm designs were then themselves folded in Mfold to predict the secondary structure of the DNAzyme itself. Those having total intramolecular energies >−1.5 kcal/mol were selected. 5 DNAzymes having intramolecular energies >−1.5 kcal/mol were selected as “sub-optimal” control DNAzymes. An additional 5 DNAzymes randomly selected but having designs consistent with the first method described above were also designed.

In Vitro Transcript Cleavage

DNAzymes designed in the second or additional subset were screened for their ability to cleave the target mRNA in vitro using the T7 promoter approach and two separate plasmids containing the sequence corresponding to the ORF of EMMPRIN. Typically, “hot” transcripts were generated using linearized plasmid DNA and the T7-system in the presence of ³²P-dUTP. The transcripts were gel purified, ethanol precipitated and resuspended in water. For the cleavage reaction, 100 nM RNA was incubated with 200 nM DNAzyme in cleavage buffer containing 1, 5 or 10 mM Mg+2 for 60 minutes at 37° C. Reaction products were separated by PAGE and imaged using a phosphoimager. Identification of the products was made possible through comparison with a ³²P-labelled RNA ladder.

Generation of Tagged Vectors for The Overexpression of EMMPRIN

Untagged and C- or N-terminal, V5 or poly-His tagged forms of EMMPRIN were generated by PCR of pcDNA3.1-EMMPRIN (Li Yan, Centocor) using the following primer pairs;

| | | |
|---|---|---|
| Construct | Forward primer 5′-3′ | Reverse primer 5′-3′ |

| | | |
|---|---|---|
| Untagged EMMPRIN | GCGCGGTACCGCCACCATGGCG | GCGCGAATTCTCAGGAAGAGTT |
| (Ox1) | GCTGCGCTGTTCGTG | CCTCTGGCG |
| | (SEQ ID NO:3) | (SEQ ID NO:4) |
| V5 epitope 5′-EMMPRIN | GCGCGGTACCGCCACCATGGGT | GCGCGAATTCTCAGGAAGAGTT |
| (Ox2) | AAGCCTATCCCTAACCCTCTCC | CCTCTGGCG |
| | TCGGTCTCGATTCTACGGCGGC | (SEQ ID NO:6) |
| | TGCGCTGTTCGTG | |
| | (SEQ ID NO:5) | |
| V5 epitope 3′-EMMPRIN | GCGCGGTACCGCCACCATGGCG | GCGCGAATTCTCACGTAGAATC |
| (Ox3) | GCTGCGCTGTTCGTG | ACCGAGGAGAGGGTTAGGGATA |
| | (SEQ ID NO:7) | GGCTTACCGGAAGAGTTCCTCT |
| | | GGCG |
| | | (SEQ ID NO:8) |
| Poly-His tag 5′-EMMPRIN | GCGCGGTACCGCCACCATGCAT | GCGCGAATTCTCAGGAAGAGTT |
| (Ox4) | CATCACCATCACCATGCGGCTG | CCTCTGGCG |
| | CGCTGTTCGTG | (SEQ ID NO:10) |
| | (SEQ ID NO:9) | |
| Poly-His tag 3′-EMMPRIN | GCGCGGTACCGCCACCATGGCG | GCGCGAATTCTCAATGGTGATG |
| (Ox5) | GCTGCGCTGTTCGTG | GTGATGATGGGAAGAGTTCCTC |
| | (SEQ ID NO:11) | TGGCG |
| | | (SEQ ID NO:12) |

The thermal cycling parameters were 1×94° C. for 3 min, 30×(94° C. for 30 s, 60° C. for 45 s, 72° C. for 2 min), 1×72° C. for 10 min. The magnesium concentration was 2 mM and Pfu polymerase (Stratagene) was used. The five resulting PCR products were cloned into the pEJC mgl ori 1 plasmid (KpnI and EcoRI sites) using standard cloning methods and the vectors were sequenced to confirm the identities of the inserts.

Results

Screening of Candidate siRNAs for Suppression of EMMPRIN Transcript

The sequences and positions of the six candidate EMMPRIN siRNAs are shown in Table 4.

TABLE 4
Sequences of candidate EMMPRIN-specific RNAs.

| | | | |
|---|---|---|---|
| | Sequence of | Position of | Placement |
| EMMPRIN | sense strand | 1^{st} base from | in |
| siRNA | 5′ to 3′ | start of cDNA | cDNA |

| 1 | GUAGGACCGGCGAGGAAUA | 33 | 5′UTR |
|---|---|---|---|
| | (SEQ ID NO:13) | | |
| 2 | GACCUUGGCUCCAAGAUAC | 149 | ORF |
| | (SEQ ID NO:14) | | |
| 3 | GUCGUCAGAACACAUCAAC | 388 | ORF |
| | (SEQ ID NO:15) | | |
| 4 | GAUCACUGACUCUGAGGAC | 478 | ORF |
| | (SEQ ID NO:16) | | |
| 5 | UGACAAAGGCAAGAACGUC | 826 | ORF |
| | (SEQ ID NO:17) | | |
| 6 | GUUGGGUUUUCUCCAUUCA | 1012 | 3′UTR |
| | (SEQ ID NO:18) | | |

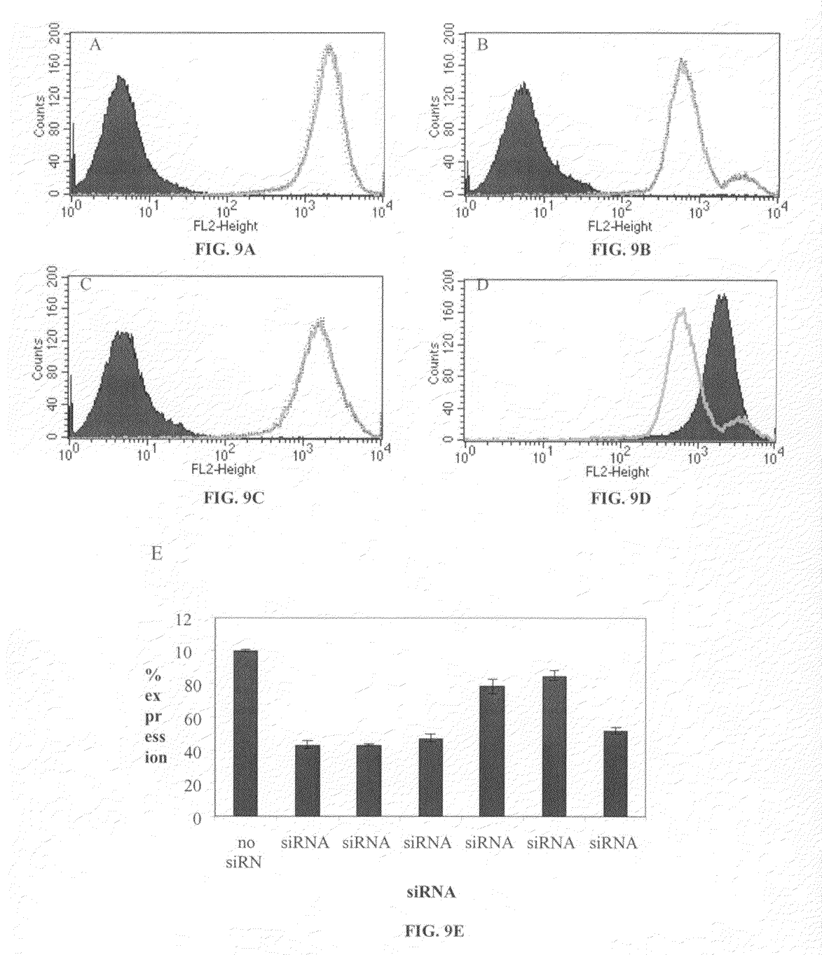

The six candidate EMMPRIN-specific siRNAs were transiently transfected into the MDA MB 231 cell line and their effects on EMMPRIN expression were monitored by antibody staining and flow cytometry at 24 and 48 hours post-transfection. The Geo Mean values for EMMPRIN were as follows: 1692 (FIG. 9A); 720 (FIG. 9B); 1403 (FIG. 9C) (average of three tests). An overlay of a representative staining profile obtained with siRNA 5 (hollow peak) and an untransfected control (solid peak) is shown in FIG. 9D. Results for all six siRNAs, represented as a percentage of the EMMPRIN expression on untransfected cells are shown in FIG. 9E (mean of triplicate samples). Suppression of EMMPRIN was effective in the cells, particularly with siRNAs 1, 2, 3 and 6 at the 48 hour time point (approximately 50% suppression; FIGS. 9A-E and Table 5).

TABLE 5
Suppression of EMMPRIN expression on MDA MB 231
cells by transfection of specific siRNAs. Values
are shown as the mean of triplicate samples.

% EMMPRIN expression

| | 24 hours post- | 48 hours post- |
|---|---|---|
| SiRNA | transfection | transfection |
| 1 | 75 ± 4.5 | 44 ± 2.2 |
| 2 | 59 ± 0.3 | 44 ± 0.8 |
| 3 | 77 ± 2.6 | 48 ± 2.4 |
| 4 | 86 ± 3.1 | 78 ± 4.5 |
| 5 | 83 ± 3.9 | 85 ± 3.1 |
| 6 | 66 ± 10.1 | 52 ± 2.0 |

Screening of Stable pSilencer EMM Clones

SiRNAs are synthetic molecules which are diluted with every cell division and so have only a transient effect on gene expression. Stable clones suppressed for EMMPRIN expression were generated by using the sequences of siRNAs 1, 2 and 6 to generate small hairpin DNA oligos and cloning them into two pSilencer vectors (Ambion). Upon transcription, the small hairpin RNAs (shRNAs) were processed into siRNAs with the same capacity of silencing genes as the synthetic molecules from which their sequences were derived. The two vectors were pSilencer 2.1, containing the U6 promoter and pSilencer 3.1 containing the H1 promoter, both with a hygromycin selectable marker (Ambion). SiRNA 3 was not used because the first transfection experiment indicated that it was not as effective at suppressing EMMPRIN as the other three siRNAs (data not shown). Three small hairpin oligos were designed according to the strategy provided by Ambion (www.ambion.com) and cloned into the two linearised pSilencer vectors to give six different constructs designated EMM1, EMM2 and EMM6.

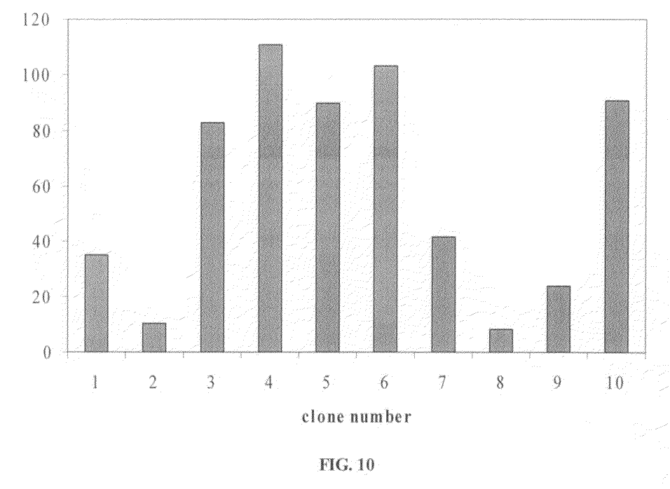

The six EMMPRIN-specific constructs and two negative controls (2.1 and 3.1) were transfected into MDA MB 231 and MDA MB 435S-GFP cells using Lipofectamine 2000 and clones were grown (expanded) under selection with hygromycin. Approximately 30 clones per construct were expanded to 6-well dishes and 10 clones per shRNA were then screened for suppression of EMMPRIN by antibody staining and flow cytometry (FIG. 10). Two or three clones showing the best levels of EMMPRIN suppression were chosen for each shRNA and expanded further. Four clones were generated in MDA MB 231 cells and three in MDA MB 435S-GFP cells in which EMMPRIN levels were suppressed by at least 80% compared to untransfected cells (Table 6).

TABLE 6
Summary of EMMPRIN expression on the surface of the
MDA MB 231 and 435S-GFP/pSilencer EMMPRIN clones.

| Construct | % EMMPRIN expression |
|---|---|
| MDA MB 231 | |
| 2.1 negative control clone 1 | 95 |
| 3.1 negative control clone 2 | 93 |
| 2.1 EMM1clone 2 | 10 |
| 2.1 EMM1clone 8 | 8 |
| 3.1 EMM2 clone 5 | 8 |
| 3.1 EMM2 clone 6 | 14 |
| 2.1 EMM6 clone 1 | 24 |
| 2.1 EMM6 clone 9 | 26 |
| MDA MB 435S-GFP | |
| 2.1 negative control clone 1 | 117 |
| 3.1 negative control clone 1 | 100 |
| 2.1 EMM1 clone 2 | 20 |
| 2.1 EMM1 clone 5 | 20 |
| 2.1 EMM1 clone 8 | 20 |
| 2.1 EMM2 clone 6 | 34 |
| 3.1 EMM2 clone 5 | 66 |
| 2.1 EMM6 clone 1 | 75 |
| 2.1 EMM6 clone 3 | 46 |

Confirmation of EMMPRIN Levels in pSilencer EMM Clones

Clones that were chosen for expansion were grown for one month in the absence of hygromycin and the levels of EMMPRIN on the cell surface of the clones was re-confirmed, not only at the protein level by antibody staining and flow cytometry and Western analysis, but also at the mRNA level by Northern blotting and Qzyme analysis which is indicative of RNAi-mediated gene silencing.

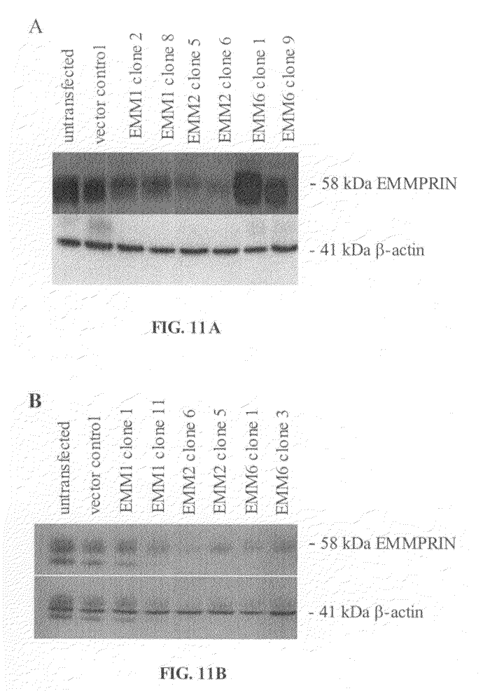

EMMPRIN expression is shown in stable pSilencer clones generated in the MDA MB 231 (FIG. 11A) and MDA MB 435S-GFP (FIG. 11B) cell lines measured by Western analysis. Cells were stably transfected with various pSilencer vectors and maintained in tissue culture for 1 month in the absence of hygromycin selection. The FIG. 11A blot was probed with EMMPRIN-specific Ab (1:2000), followed by a goat anti-mouse HRP-conjugate (1:2000). The FIG. 11B blot was probed with 91-actin-specific antibody (1:5000), followed by a goat anti-mouse HRP-conjugate (1:2000) as a loading control.

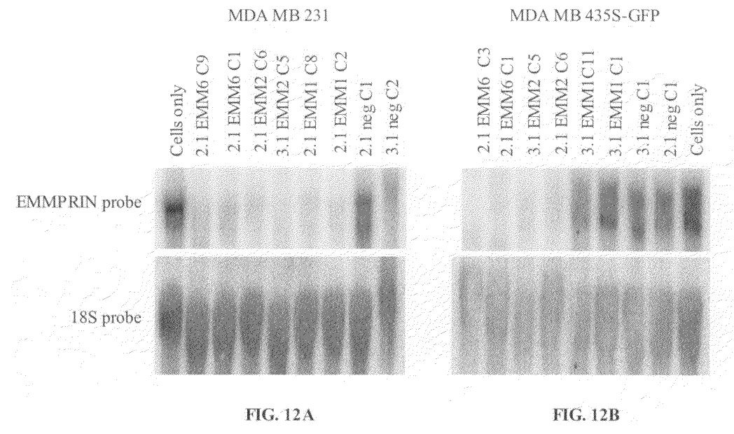

EMMPRIN expression is shown in stable pSilencer clones generated in the MDA MB 231 (FIG. 12A) and MDA MB 435S-GFP (FIG. 12B) cell lines measured by northern analysis. RNA (20 pg) was probed with 50 ng of a labelled EMMPRIN PCR product (FIG. 12A blots). The membranes were stripped and reprobed with a labelled 18S probe as a loading control (FIG. 12B blots).

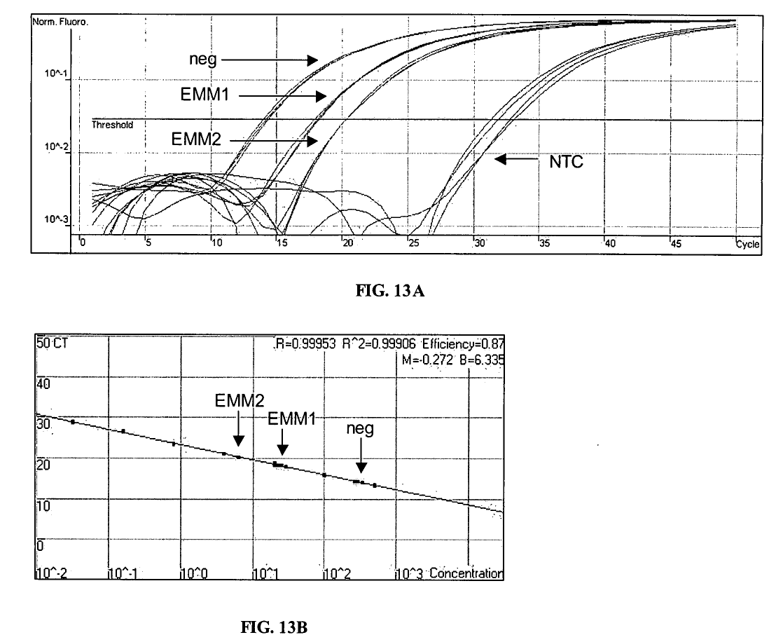

FIGS. 13A and B show the levels of the EMMPRIN transcript in MDA MB 231 clones containing the pSilencer negative control (neg) or the EMMPRIN-specific shRNA vector (EMM1 and EMM2). These levels were measured relative to the levels of the EMMPRIN transcript in dilutions of RNA from the untransfected MDA MB 231 cells used to generate a standard curve (FIG. 13B). A total of 250 ng of total RNA was used for the test samples to ensure that each value fit within the standard curve. Standards were run in duplicate, test samples in triplicate and no template controls (NTC) in quadruplicate.

There was a good correlation between the levels of EMMPRIN transcript measured by flow cytometry, Northern blotting and Qzyme (Table 4). The data also demonstrated that the levels of EMMPRIN on the cell surface of the clones were stable over time even in the absence of hygromycin selection (comparing Table 6 and Table 7) and even after freeze-thawing.

TABLE 7
Confirmation of EMMPRIN suppression on the surface of selected
MDA MB 231/pSilencer clones by flow cytometry, Northern analysis
and Qzyme at least one month after initial screening.

| | % relative EMMPRIN expression | | | |
|---|---|---|---|---|
| | MDA MB | 3.1 | 2.1 | 3.1 |
| Assay | 231 | Neg C2 | EMM1 C2 | EMM2 C5 |
| Flow cytometry | 100 | 95 | 12 | 12 |
| Northern | 100 | 50 | 8 | 5 |
| Qzyme | 100 | 84 | 9 | 7 |

Screening of EMMPRIN-Targeted DNAzymes

The sequences of the twenty candidate EMMPRIN-targeted DNAzymes are shown below in Table 8.

TABLE 8

Sequences of the initial set of twenty DNAzymes
designed to target EMMPRIN.

| | | | |
|---|---|---|---|
| | | Position of | Placement |
| DNAzyme | Sequence 5′ to 3′ | last base from | in |
| Name | 5′ to 3′ | start of cDNA | cDNA |

| | | | |
|---|---|---|---|
| | TGATTCCTAGGCTAGCTA | 0 | ′UTR |
| | CAACGATCCTCGCCG | | |
| | (SEQ ID NO:19) | | |
| | CAGCGCGAAGGCTAGCTA | 8 | RF |
| | CAACGACCCAGCAGC | | |
| | (SEQ ID NO:20) | | |
| 2 | TGAGGAGTAGGCTAGCTA | 57 | RF |
| | CAACGACTTGGAGCC | | |
| | (SEQ ID NO:21) | | |
| 5 | TCAGCACCAGGCTAGCTA | 26 | RF |
| | CAACGAGCCCCCCTT | | |
| | (SEQ ID NO:22) | | |
| 3 | GGAGCTGGAGGCTAGCTA | 43 | RF |
| | CAACGAGTTGGCCGT | | |
| | (SEQ ID NO:23) | | |
| 8 | CCTCGTTGAGGCTAGCTA | 94 | RF |
| | CAACGAGTGTTCTGA | | |
| | (SEQ ID NO:24) | | |
| 6 | AGTCAGTGAGGCTAGCTA | 72 | RF |
| | CAACGACTTGTACCA | | |
| | (SEQ ID NO:25) | | |
| 3 | CGGCCTCCAGGCTAGCTA | 74 | RF |
| | CAACGAGTTCAGGTT | | |
| | (SEQ ID NO:26) | | |
| 7 | GGAGCGTGAGGCTAGCTA | 40 | RF |
| | CAACGAGATGGCCTG | | |
| | (SEQ ID NO:27) | | |
| 1 | GCACCAGCAGGCTAGCTA | 03 | RF |
| | CAACGACTCAGCCAC | | |
| | (SEQ ID NO:28) | | |
| 0 | CCTTTGTCAGGCTAGCTA | 18 | RF |
| | CAACGATCTGGTGCT | | |
| | (SEQ ID NO:29) | | |
| 2 | CTCGGGCCAGGCTAGCTA | 64 | RF/3′UTR |
| | CAACGACTGCCTCAG | | |
| | (SEQ ID NO:30) | | |
| 8 | GGGAATCTAGGCTAGCTA | 67 | ′UTR |
| | CAACGAGGGGTGGGT | | |
| | (SEQ ID NO:31) | | |
| 6 | CCTAAGGAAGGCTAGCTA | 032 | ′UTR |
| | CAACGAAGAATCCTG | | |
| | (SEQ ID NO:32) | | |
| 3 | CGGCCATGAGGCTAGCTA | 125 | ′UTR |
| | CAACGATCAGACCCA | | |
| | (SEQ ID NO:33) | | |
| 8 | GGGCAAGGAGGCTAGCTA | 178 | ′UTR |
| | CAACGACTGGCACTG | | |
| | (SEQ ID NO:34) | | |
| 05 | AGTCGAACAGGCTAGCTA | 305 | ′UTR |
| | CAACGAAGACCCGTG | | |
| | (SEQ ID NO:35) | | |
| 07 | AGGCTGTGAGGCTAGCTA | 382 | ′UTR |
| | CAACGAGGGGTCGCC | | |
| | (SEQ ID NO:36) | | |
| 11 | GCCCCCGGAGGCTAGCTA | 425 | ′UTR |
| | CAACGAGCACAGACA | | |
| | (SEQ ID NO:37) | | |
| 14 | CTGGTGCTAGGCTAGCTA | 533 | ′UTR |
| | CAACGAAGAGAGCCG | | |
| | (SEQ ID NO:38) | | |

Prior to delivery, transfection conditions were optimized by use of a FAM-labelled DNAzyme (Table 9).

TABLE 9
Optimisation of FAM-labelled DNAzyme transfection
of MDA MB 231 cells using lipofectamine.

| | | 6-well plate | 60 mm dish |
|---|---|---|---|
| DNAzyme/ | | % transfection | % transfection |
| μg | Lipofectamine/μl | efficiency | efficiency |
| 0 | 8 | 0.7 | 0.2 |
| 1 | 3 | 57.2 | 36.6 |
| 3 | 8 | 95.2 | 71.9 |
| 6 | 15 | 99.3 | 97.9 |
| 20 | 40 | 99.7 | 99.6 |
| 48 | 96 | 99.2 | 99.0 |

MDA MB 231 cells were then transiently transfected in 6-well plates with the EMMPRIN-specific DNAzymes, using 3 μg of DNAzyme and 8 μl of lipofectamine as these conditions gave both a high transfection efficiency and low cell toxicity. EMMPRIN expression on the transfected cells was measured by antibody staining and flow cytometry, but no effective DNAzyme was identified.

Repeated Screening of EMMPRIN-Targeted DNAzymes

The sequences of the further 15 candidate EMMPRIN-targeted DNAzymes are shown below in Table 10.

TABLE 10

Sequences of the second set of fifteen
DNAzymes designed to target EMMPRIN.

| | | Position of first |
|---|---|---|
| DNAzyme | | base from start |
| Name | Sequence 5′ to 3′ | codon |
| 1482 | CCTTCAGCAGGCTAGCTACAACGA | 172 |
| | CACGCCCCC | |
| | (SEQ ID NO:39) | |
| 1483 | CGGAGTCCAGGCTAGCTACAACGA | 217 |
| | CTTGAACTC | |
| | (SEQ ID NO:40) | |
| 1484 | CGGCCTTCAGGCTAGCTACAACGA | 310 |
| | TCTGGGAGG | |
| | (SEQ ID NO:41) | |
| 1485 | CAGCCACGAGGCTAGCTACAACGA | 634 |
| | GCCCAGGAA | |
| | (SEQ ID NO:42) | |
| 1486 | GCAGGAGTAGGCTAGCTACAACGA | 243 |
| | TCTCCCCAC | |
| | (SEQ ID NO:43) | |
| 1487 | GCGCTGTCAGGCTAGCTACAACGA | 122 |
| | TCAAGGAGC | |
| | (SEQ ID NO:44) | |
| 1488 | GCCCTGTGAGGCTAGCTACAACGA | 139 |
| | CTCTGTGGC | |
| | (SEQ ID NO:45) | |
| 1489 | TAGCTCTGAGGCTAGCTACAACGA | 489 |
| | CGGCCCTGC | |
| | (SEQ ID NO:46) | |
| 1491 | ACTTGCAGAGGCTAGCTACAACGA | 364 |
| | CAGCATGGC | |
| | (SEQ ID NO:47) | |
| 1492 | TGACCAGCAGGCTAGCTACAACGA | 652 |
| | CAGCACCTC | |
| | (SEQ ID NO:48) | |
| 1493 | TGAGGAGTAGGCTAGCTACAACGA | 100 |
| | CTTGGAGCC | |
| | (SEQ ID NO:49) | |
| 1494 | CCGTGCCCAGGCTAGCTACAACGA | 271 |
| | GGGCTCGGG | |
| | (SEQ ID NO:50) | |
| 1495 | CCTCGTTGAGGCTAGCTACAACGA | 337 |
| | GTGTTCTGA | |
| | (SEQ ID NO:51) | |
| 1496 | AGACCAGCAGGCTAGCTACAACGA | 358 |
| | GGCCGTCTC | |
| | (SEQ ID NO:52) | |
| 1497 | CGGCCATGAGGCTAGCTACAACGA | 595 |
| | TCAGACCCA | |
| | (SEQ ID NO:53) | |

Of these, 6 (1487-1492) were predicted to be optimal based on thermodynamic predictions from the GENBANK sequence. However, sequencing of two plasmid EMMPRIN clones used for the cleavage analysis revealed slight differences in sequence relative to the GENBANK sequence. This had repercussions on predictions of folding according to Oligowalk. As a result, only 1487, 1488 and 1492 were then predicted to be optimal for the generated transcripts. With 10 mM Mg⁺², strong cleavage of the T7 substrates generated from the two clones was only observed for DNAzyme 1488, with the formation of product bands at the expected size on the PAGE gel. Slight cleavage of clone “7” was seen with DNAzymes 1508, 1509 and 1512. With clone “4,” slight cleavage was present in incubations with 1509 and 1512. Significant cleavage of both clone transcripts was also observed when the Mg⁺² was titrated down to 1 and 5 mM.

The cleavage activity remained strong when variants of 1488, which were synthesized with chemical modifications designed to improve stability in cells, were tested under identical conditions.

| | | |
|---|---|---|
| DZ | Sequence 5′ to 3′ | Comment |

| | | |
|---|---|---|
| 1488 | GCCCTGTGAGGCTAGCTACAACGACTC | Unmodified PO |
| | TGTGGC | |
| | (SEQ ID NO:45) | |
| 1519 | GCCCTGTGAGGCTAGCTACAACGACTC | Inverted base |
| | TGTGGC [3′-3′T] | |
| | (SEQ ID NO:54) | |
| 1520 | GCCCTGTGAGGCTAGCTACAACGACTC | Inverted base |
| | TGTGGC [3′-3′G] | |
| | (SEQ ID NO:55) | |
| 1521 | GCCCTGTGAGGCTAGCTACAACGACTC | Inverted base |
| | TGTGGC [3′-3′C] | |
| | (SEQ ID NO:56) | |
| 1519b | G*C*CCTGTGAGGCTAGCTACAACGAC | Inverted base |
| | TCTGTGG*C* [3′-3′T] | 2 + 2 2′Omethyl |
| | (SEQ ID NO:57) | |
| 1520b | G*C*CCTGTGAGGCTAGCTACAACGAC | Inverted base |
| | TCTGTGG*C* [3′-3′G] | 2 + 2 2′Omethyl |
| | (SEQ ID NO:58) | |

Overexpression of EMMPRIN

Tagged and untagged EMMPRIN were cloned into the pEJC mgl ori 1 episomal plasmid, in which EMPRIN expression is under the control of the CMV promoter. The five EMMPRIN-expressing vectors and an EMMPRIN negative control plasmid were transfected into MDA MB 231 cells by electroporation and clones were selected with puromycin.

The pEGFP-N1 plasmid was also transfected into the cells so that the transfection efficiency could be determined. GFP expression was measured at 24- and 96-hours post-transfection and the transfection efficiency was found to be 45% at both time points (data not shown), demonstrating that the episome was stable over a 96-hour period even in the absence of selection by puromycin. The following table shows the number of clones picked for each construct and the number of clones that survived to be expanded into T75s.

TABLE 11
Survival of EMMPRIN overexpression clones.

| Construct | No. of clones picked | No. of clones survived |
|---|---|---|
| pEJC mgl ori 1 | 12 | 7 |
| Ox1 | 20 | 6 |
| Ox2 | 24 | 6 |
| Ox3 | 18 | 10 |
| Ox4 | 9 | 4 |
| Ox5 | 20 | 8 |

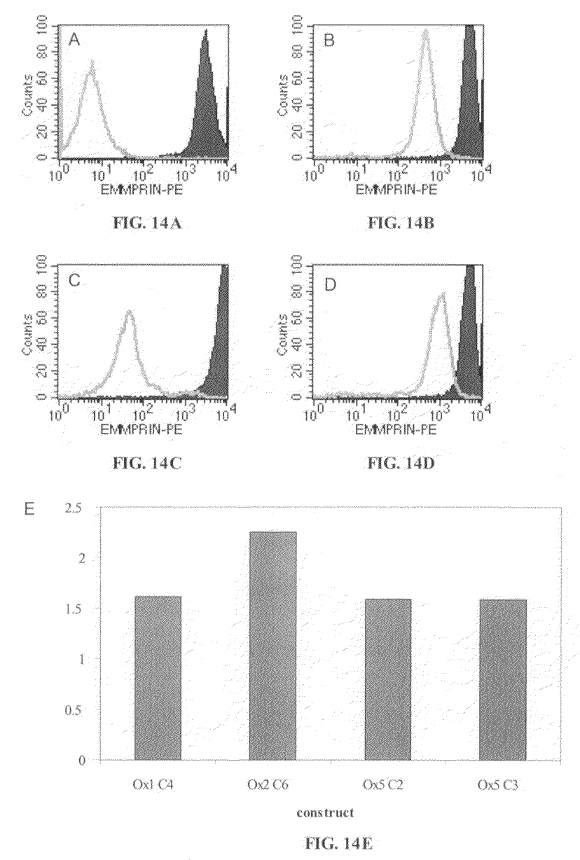

The number of clones that formed after transfection with the overexpression constructs was considerably less than with the pSilencer constructs. Furthermore, a substantial proportion of the clones that were picked did not survive and could not be expanded. The clones that did survive were surface stained for EMMPRIN expression and analysed by flow cytometry. The results for clones transfected with the Ox1, Ox4 and Ox5 constructs are shown in FIGS. 14A-E. Geo Mean values for EMMPRIN (solid peaks) are as follows: untransfected cells, 2645 (FIG. 14A); Ox1 clone 4, 4273 (FIG. 14B); Ox2 clone 6, 5966 (FIG. 14C); Ox5 clone 3, 4199 (FIG. 14D), with the results consolidated in FIG. 14E. Hollow peaks denote GFP expression (GFP encoded on the pEJC mgl ori 1 plasmid). None of the clones transfected with the Ox 3 and Ox 4 plasmids were found to have increased levels of surface EMMPRIN expression compared to untransfected control cells.

The highest level of EMMPRIN overexpression was found on the surface of clones Ox1 clone 4, Ox2 clone 6 and Ox5 clones 2, 3 and 4, and was found to be 1.5 to 2-fold higher than on untransfected cells. It is possible that a greater than two-fold increase in EMMPRIN expression could not be achieved because clones that highly overexpressed EMMPRIN were not able to survive.

EMMPRIN Expression in Colorectal Cancer Cell Lines

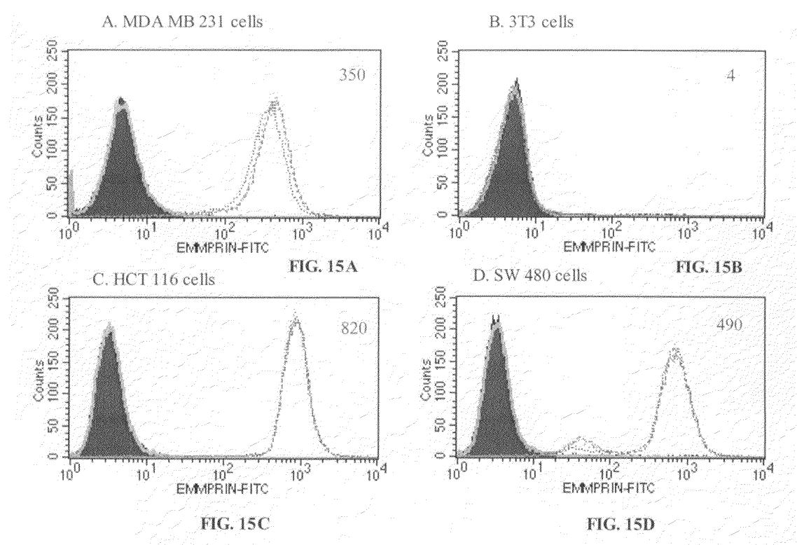

The level of EMMPRIN expression in colorectal cancer cell lines has not been reported in the literature. In fact, there are no reported investigations of the role of EMMPRIN in colorectal cancer. The levels of EMMPRIN found on the surface of several colorectal cancer cell lines was therefore examined by antibody staining and flow cytometry. The results (FIGS. 15A-D and 16A-F) show that EMMPRIN was detectable on the surface of all of the colorectal cancer cell lines tested and that EMMPRIN expression was particularly high on the surface of the HCT116 and KM12SM cell lines. No correlation has yet been found between the level of EMMPRIN expression and the metastatic potential of the cells.

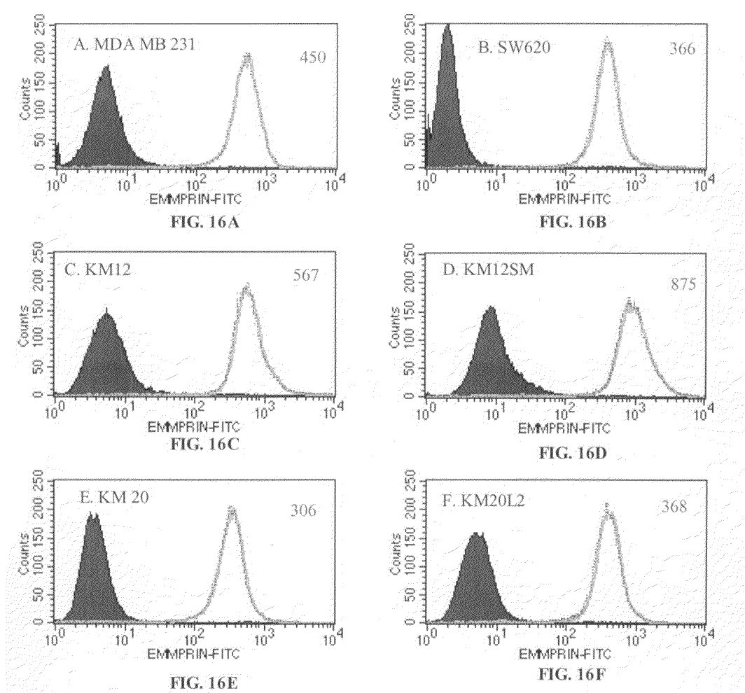

Specifically, in FIGS. 15A-D, isotype control antibody results are shown by solid and hollow peaks. Dashed lines represent EMMPRIN-specific antibody. 3T3 cell line is a mouse fibroblast line and was included as a negative control. The Geo Mean using the EMMPRIN-specific antibody is shown as part of the broken lines (mean of three samples). In FIGS. 16A-F, isotype control antibody results are shown by solid peaks. Dashed and hollow peaks represent EMMPRIN-specific antibody. The Geo Mean using the EMMPRIN-specific antibody is shown as part of the broken lines around the hollow peaks (mean of three samples).

Effect of EMMPRIN shRNA on VEGF Expression

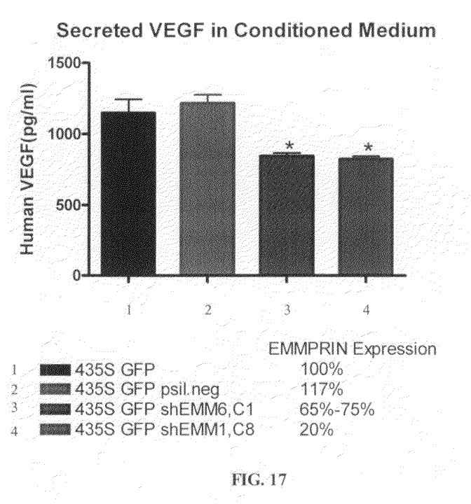

Human breast cancer cells MDA-MB-435S GFP were stably transformed with various EMMPRIN shRNA expression constructs. The effect of EMMPRIN expression on VEGF production was analyzed by an ELISA assay. As shown in FIG. 17, EMMPRIN levels were inhibited to 65-75% or 20% of the parental in shEMM6, C1 and shEMM1, C8 clones, respectively. Concomitantly, VEGF production was significantly inhibited as the consequence of EMMPRIN inhibition. Sample 1 in the bar chart and legend corresponds to the control, sample 2 the pSilencer negative control, sample 3 the shEMM6, C1, and sample 4 the shEMM1, C8.

It will be clear that the invention can be practiced otherwise than as particularly described in the foregoing description and examples. Numerous modifications and variations of the present invention are possible in light of the above teachings and, therefore, are within the scope of the appended claims.

### REFERENCES

the following references and other references cited herein are incorporated by reference.
Biswas C, Zhang Y, DeCastro R, Guo H, Nakamura T, Kataoka H, Nabeshima K: The human tumor cell-derived collagenase stimulatory factor (renamed EMMPRIN) is a member of the immunoglobulin superfamily. Cancer Res 1995; 55:434-9.
Bordador, L. C., Li, X., Toole, B., Chen, B., Regezi, J., Zardi, L., Hu, Y. & Ramos, D. M. (2000). Expression of emmprin by oral squamous cell carcinoma. Int J Cancer 85, 347-52.
Brooks et al., Science, Vol. 264, (1994), 569-571.
Caudroy, S., Polette, M., Tournier, J. M., Burlet, H., Toole, B., Zucker, S. & Birembaut, P. (1999). Expression of extracellular matrix metalloproteinase inducer (EMMPRIN) and the matrix metalloproteinase-2 in bronchopulmonary and breast lesions. Journal of Histochemistry and Cytochemistry 47, 1575-80.
Cohen, et al. J. Biol. Chem. 271: 736-741, (1996).
Coussens L M, Fingleton B, Matrisian L M: Matrix metalloproteinase inhibitors and cancer: trials and tribulations. Science 2002; 295:2387-92.
Deryugina El, Soroceanu L, Strongin A Y: Up-regulation of vascular endothelial growth factor by membrane-type 1 matrix metalloproteinase stimulates human glioma xenograft growth and angiogenesis. Cancer Res 2002; 62:580-8.
Eliceiri, et al., J. Clin. Invest.103: 1227-1230 (1999).
Elliott et al., Lancet 344:1125-1127 (1994).
Ellis S M, Nabeshima K, Biswas C: Monoclonal antibody preparation and purification of a tumor cell collagenase-stimulatory factor. Cancer Res 1989; 49:3385-91.
Folkman J: Angiogenesis in cancer, vascular, rheumatoid and other disease. Nat Med 1995; 1:27-31.
Folkman and Cotran, Relation of vascular proliferation to tumor growth, Int. Rev. Exp. Pathol.'16, 207-248 (1976)
Folkman J., J. Invest. Dermatol., 59, 40-48 (1972).
Folkman, et al. N Engl J Med 285: 1181-1186, (1971).
Folkman, et al. N Engl J Med 333: 1757-1763, (1995).
Friedlander et al., Science 270: 1500-1502 (1995).
Giurguis, R., Javadpour, N., Sharareh, S., Biswas, C. & el-Amin, W. (1990). A new method for evaluation of urinary autocrine motility factor and tumour cell collagenase stimulating factor as markers for urinary tract cancers. Journal of Occupational Medicine 32, 846-53.
Guo H, Zucker S, Gordon M K, Toole B P, Biswas C: Stimulation of matrix metalloproteinase production by recombinant extracellular matrix metalloproteinase inducer from transfected Chinese hamster ovary cells. J Biol Chem 1997; 272:24-7.
Guo, H., Majmuda, G., Jensen, T. C., Biswas, C., Toole, B. P. & Gordon, M. K. (1998). Characterization of the gene for the human EMMPRIN, a tumor cell surface inducer of matrix metalloproteinases. Gene 220, 99-108.
Haseneen, N. A., Vaday, G. G., Zucker, S. & Foda, H. D. (2003). Mechanical stretch induces MMP-2 release and activation in lung endothelium: role of EMMPRIN. American journal of Physiology Lung Cell Molecular Physiology 284, L541-547.
Heslop, H H, Benaim, E, Brenner, M K, Krance, R A, Stricklin, L M, Rochester, R J, Billing R. Response of storoid—resistant graft-versus-host disease to lymphoblast antibody CBL1. Lancet 1995; 346:805-06.
Javadpour N, Guirguis R: Tumor collagenase-stimulating factor and tumor autocrine motility factor as tumor markers in bladder cancer—an update. Eur Urol 1992; 21 Suppl 1:1-4.
Kaname, T., Miyauchi, T., Kuwano, A., Muramatsu, T. & Kajii, T. (1993). Mapping basigin (BSG), a member of the immunoglobulin superfamily, to 19p13.3. Cytogenet. Cell Genet. 64, 195-197.
Kataoka H, DeCastro R, Zucker S, Biswas C: Tumor cell-derived collagenase-stimulatory factor increases expression of interstitial collagenase, stromelysin, and 72-kDa gelatinase. Cancer Res 1993; 53:3154-8.
Klagsbrun M, Moses M A: Molecular angiogenesis. Chem Biol 1999; 6:R217-24.
Koch, C, Staffler, G, Huttinger, R, Hilgert, I, Prager, E, Cerny, J, Steinlein, P, Majdic, O, Horejsi, V. and Stockinger, H. T cell activation-associated epitopes of C147 in regulation of the T cell response, and their definition by antibody affinity and antigen density. Internat. Immunol. 1999; 11:777-786.
Koch A K, Polyerini P J and Leibovich S J, Arth; 15 Rhenium, 29, 471-479 (1986)
Koch A K, Arthritis Rheum, 41, 951 962 (1998).
Li, T. F., Santavirta, S., Virtanen, I., Kononen, M., Takagi, M. & Konttinen, Y. T. (1999). Increased expression of EMMPRIN in the tissue around loosened hip prostheses. Acta Orthop Scand 70, 446-51.
Liu, C., Cheng, R., Sun, L. Q. & Tien, P. (2001). Suppression of platelet-type 12-lipoxygenase activity in human erythroleukemia cells by an RNA-cleaving DNAzyme. Biochem Biophys Res Commun 284, 1077-1082.
Major, T. C., Liang, L., Lu, X., Rosebury, W. & Bocan, T. M. (2002). Extracellular matrix metalloproteinase inducer (EMMPRIN) is induced upon monocyte differentiation and is expressed in human atheroma. Arteriosclerosis and Thrombosis Vascular Biology 22, 1200-1207.
Milstein and Cuello, Nature 305:537 (1983).
Muraoka K, Nabeshima K, Murayama T, Biswas C, Koono M: Enhanced expression of a tumor-cell-derived collagenase-stimulatory factor in urothelial carcinoma: its usefulness as a tumor marker for bladder cancers. Int J Cancer 1993; 55:19-26.
Norgauer, J., Hildenbrand, T., Idzko, M., Panther, E., Bandemir, E., Hartmann, M., Vanscheidt, W. & Herouy, Y. (2002). Elevated expression of extracellular matrix metalloproteinases inducer (CD147) and membrane-type matrix metalloproteinases in venous leg ulcers. British Journal of Dermatology 147, 1180-1186.
O'Reilly et. al. Cell 79: 315-328.
Polette M, Gilles C, Marchand V, Lorenzato M, Toole B, Tournier J M, Zucker S, Birembaut P: Tumor collagenase stimulatory factor (TCSF) expression and localization in human lung and breast cancers. J Histochem Cytochem 1997; 45:703-9.
Sambrook, J., Fritsch, E. F. & Maniatis, T. (1989). Molecular cloning: A laboratory manual, 2nd edition edn. New York: Cold Spring Harbour.
Sameshima T, Nabeshima K, Toole B P, Yokogami K, Okada Y, Goya T, Koono M, Wakisaka S: Expression of emmprin (CD147), a cell surface inducer of matrix metalloproteinases, in normal human brain and gliomas. Int J Cancer 2000a; 88:21-7.
Sameshima T, Nabeshima K, Toole B P, Yokogami K, Okada Y, Goya T, Koono M, Wakisaka S: Glioma cell extracellular matrix metalloproteinase inducer (EMMPRIN) (CD147) stimulates production of membrane-type matrix metalloproteinases and activated gelatinase A in co-cultures with brain-derived fibroblasts. Cancer Lett 2000b; 157:177-84.
Santoro, S. W. & Joyce, G. F. (1997). A general purpose RNA-cleaving DNA enzyme. Proceedings of the National Academy of Sciences USA 94, 4262-4266.
Santoro, S. W. & Joyce, G. F. (1998). Mechanism and utility of an RNA-cleaving DNA enzyme. Biochemistry 37, 13330-13342.
Sounni N E, Devy L, Hajitou A, Frankenne F, Munaut C, Gilles C, Deroanne C, Thompson E W, Foidart J M, Noel A: MT1-MMP expression promotes tumor growth and angiogenesis through an up-regulation of vascular endothelial growth factor expression. Faseb J 2002; 1 6:555-64.
Staffler, G, Szekeres, A, Schütz, G H, Säemann, M D, Prager, E, Zeyda, M, Drbal, K, Zlabinger, G J, Stulnig, T M, and Stockinger, H. Selective inhibition of T cell activation via CD147 through novel modulation of lipid rafts. J. Immunol. 2003: 171:1707-1714.
Stupack D G, Storgard C M and Cheresh D A, Braz. J. Med. Biol. Res., 32, 578-581 (1999).
Suresh et al., Methods in Enzymology 121:210 (1986).
Tang, Y, Kesavan, P, Nakada, M T, Yan, L. Tumor-stroma interaction: Positive feedback of extracellular matrix metalloproteinase inducer (EMMPRIN) expression and matrix metalloproteinase-dependent generation of soluble EMMPRIN. Mol. Cencer. Res. 2004; 2:73-80.
Thorns, C., Feller, A. C. & Merz, H. (2002). EMMPRIN (CD147) is expressed in Hodgkin's lymphoma and anaplastic large cell lymphoma. An immunohistochemical study of 60 cases. Anticancer Research 22, 1983-1986.
Todd, A. V., Fuery, C. J., Impey, H. L., Applegate, T. L. & Haughton, M. A. (2000). DzyNA-PCR: Use of DNAzymes to detect and quantify nucleic acid sequences in a real-time fluorescent format. Clinical Chemistry 46, 625-630.
Traunecker et al., EMBO J. 10:3655 (1991).
van der Oord, J. J., Paemen, L., Opdenakker, G. & de Wolf-Peeters, C. (1997). Expression of gelatinase B and the extracellular matrix metalloproteinase inducer EMMPRIN in benign and malignant pigment cell lesions of the skin. American Journal of Pathology 151, 665-70.
Yamani, M. H., Starling, R. c., Young, J. B., Cook, D., Yu, Y., Vince, D. G., McCarthy, P. & Ratliff, N. B. (2002). Acute vascular rejection is associated with up-regulation of vitronectin receptor (alphavbeta3), increased expression of tissue factor, and activation of the extracellular matrix metalloproteinase induction system. Journal of Heart and Lung Transplant 21, 983-989.
Zhang, X., Chen, Z., Huang, H., Gordon, J. R. & Xiang, J. (2002). DNA microarray analysis of the gene expression profiles of naive versus activated tumor-specific T cells. Life Sciences 71, 3005-3017.
Zucker, S., Cao, J. & Chen, W.-T. (2000). Critical appraisal of the use of matrix metalloproteinase inhibitors in cancer treatment. Oncogene 19, 6642-6650.
Zucker S, Hymowitz M, Rollo E E, Mann R, Conner C E, Cao J, Foda H D, Tompkins D C, Toole B P: Tumorigenic potential of extracellular matrix metalloproteinase inducer. Am J Pathol 2001; 158:1921-8.

1. A method for treating an angiogenesis-dependent disease in a mammal in need thereof comprising administering to the mammal a nucleic acid based EMMPRIN antagonist in an amount effective to inhibit angiogenesis in said mammal.

2. The method according to claim 1, wherein the EMMPRIN antagonist is an siRNA molecule.

3. The method according to claim 1, wherein the EMMPRIN antagonist is an shRNA molecule.

4. The method according to claim 1, wherein the EMMPRIN antagonist is a DNAzyme molecule.

5. The method according to claim 1, wherein the EMMPRIN antagonist is an antisense molecule.

6. The method according to any one of claims 2-5, wherein the EMMPRIN antagonist is administered from at least one mode selected from the group consisting of intravenous, oral, rectal, transmucosal, intestinal, intramuscular, subcutaneous, intramedullar, intrathecal, direct intraventricular, intraperitoneal, intraocular, intravesicular instillation, intranasal, and inhalation.

7. The method according to any one of claims 2-5, wherein the EMMPRIN antagonist is administered along with an anti-EMMPRIN antibody.

8. The method according to claim 1, wherein the mammal is a human patient.

9. The method according to claim 1, wherein the angiogenesis-dependent disease is cancer.

10. The method according to claim 1, wherein the angiogenesis-dependent disease is a disease selected from the group consisting of angioma, angiofibroma, diabetic retinopathy, premature infant's retinopathy, neovascular glaucoma, corneal disease induced by angiogenesis, involutional macula, macular degeneration, pterygium, retinal degeneration, retrolental fibroplasias, granular conjunctivitis, psoriasis, telangiectasis, pyogenic granuloma, seborrheic dermatitis, acne and arthritis.

11. The method according to claim 1, wherein said angiogenesis dependent disease is an inflammatory disease selected from the group consisting of rheumatoid arthritis, macular degeneration, psoriasis, and diabetic retinopathy.

12. The method according to claim 1, wherein said angiogenesis dependent disease is an angiogenic skin disorder selected from the group consisting of psoriasis, venous ulcers, acne, rosacea, warts, eczema, hemangiomas, and lymphangiogenesis.

13. The method according to claim 1, wherein said angiogenesis dependent disease is a disorder involving corneal or retinal neovascularization.

14. A method for inhibiting tumor growth in a mammal in need thereof comprising administering to the mammal a nucleic acid based EMMPRIN antagonist in an amount effective to inhibit angiogenesis of the vasculature supporting the growth of said tumor.

15. A method for preventing tumor growth in a mammal in need thereof comprising administering to the mammal a nucleic acid based EMMPRIN antagonist in an amount effective to inhibit angiogenesis of the vasculature supporting the growth of said tumor.

16. A method for preventing metastases in a mammal in need thereof comprising administering to the mammal a nucleic acid based EMMPRIN antagonist in an amount effective to prevent metastases in said mammal.

17. The method according to any one of claims 1-5, 14, 15, or 16, wherein the EMMPRIN antagonist is administered in combination with a second anti-angiogenic agent.

18. An antagonist to EMMPRIN comprising a molecule selected from the group consisting of an siRNA, shRNA, antisense, and DNAzyme molecule.

19. The antagonist according to claim 18, wherein the antagonist is effective in treating an angiogenesis-dependent disease in a mammal.

20. The antagonist according to claim 18, wherein the antagonist is an siRNA molecule selected from SEQ ID NOS: 13-18.

21. The antagonist according to claim 18, wherein the antagonist is a DNAzyme selected from the group consisting of SEQ ID NOS: 39-58.

22. The antagonist according to claim 21, wherein the antagonist is a DNAzyme comprising SEQ ID NO:54 and nucleotide 34 of SEQ ID NO:54 is a modified base with an inverted [3′-3′] linkage.

23. The antagonist according to claim 21, wherein the antagonist is a DNAzyme comprising SEQ ID NO: 55 and nucleotide 34 of SEQ ID NO: 55 is a modified base with an inverted [3′-3′] linkage.

24. The antagonist according to claim 21, wherein the antagonist is a DNAzyme comprising SEQ ID NO: 56 and nucleotide 34 of SEQ ID NO:56 is a modified base with an inverted [3′-3′] linkage.

25. The antagonist according to claim 21, wherein the antagonist is a DNAzyme comprising SEQ ID NO: 57 and nucleotide 34 of SEQ ID NO: 57 is a modified base with an inverted [3′-3′] linkage and nucleotides 1, 2, 32, and 33 of SEQ ID NO:57 are modified 2′ o-methyl nucleotides.

26. The antagonist according to claim 21, wherein the antagonist is a DNAzyme comprising SEQ ID NO: 58 and nucleotide 34 of SEQ ID NO: 58 is a modified base with an inverted [3′-3′] linkage and nucleotides 1, 2, 32, and 33 in SEQ ID NO: 57 are modified 2′ o-methyl nucleotides.

27. Any invention described herein.


SEQUENCE LISTING (58 sequences)

SEQ ID NO: 1 - DNA, 55 bp, Artificial Sequence
  Primer
cagcacaacctcgttgtagctagcctcaccaaccgtgaagtcgtcagaacacatc

SEQ ID NO: 2 - DNA, 20 bp, Artificial Sequence
  Primer
agagtcagtgatcttgtacc

SEQ ID NO: 3 - DNA, 37 bp, Artificial Sequence
  Primer
gcgcggtaccgccaccatggcggctgcgctgttcgtg

SEQ ID NO: 4 - DNA, 31 bp, Artificial Sequence
  Primer
gcgcgaattctcaggaagagttcctctggcg

SEQ ID NO: 5 - DNA, 79 bp, Artificial Sequence
  Primer
gcgcggtaccgccaccatgggtaagcctatccctaaccctctcctcggtctcgattctacggcggctgcgctgttcgtg

SEQ ID NO: 6 - DNA, 31 bp, Artificial Sequence; identical to SEQ ID NO: 4
  Primer

SEQ ID NO: 7 - DNA, 37 bp, Artificial Sequence; identical to SEQ ID NO: 3
  Primer

SEQ ID NO: 8 - DNA, 70 bp, Artificial Sequence
  Primer
gcgcgaattctcacgtagaatcaccgaggagagggttagggataggcttaccggaagagttcctctggcg

SEQ ID NO: 9 - DNA, 55 bp, Artificial Sequence
  Primer
gcgcggtaccgccaccatgcatcatcaccatcaccatgcggctgcgctgttcgtg

SEQ ID NO: 10 - DNA, 31 bp, Artificial Sequence; identical to SEQ ID NO: 4
  Primer

SEQ ID NO: 11 - DNA, 37 bp, Artificial Sequence; identical to SEQ ID NO: 3
  Primer

SEQ ID NO: 12 - DNA, 49 bp, Artificial Sequence
  Primer
gcgcgaattctcaatggtgatggtgatgatgggaagagttcctctggcg

SEQ ID NO: 13 - RNA, 19 nt, Artificial Sequence
  Whereas Artificial Sequence is comprised of siRNA.
guaggaccggcgaggaaua

SEQ ID NO: 14 - RNA, 19 nt, Artificial Sequence
  Whereas Artificial Sequence is comprised of siRNA.
gaccuuggcuccaagauac

SEQ ID NO: 15 - RNA, 19 nt, Artificial Sequence
  Whereas Artificial Sequence is comprised of siRNA.
gucgucagaacacaucaac

SEQ ID NO: 16 - RNA, 19 nt, Artificial Sequence
  Whereas Artificial Sequence is comprised of siRNA.
gaucacugacucugaggac

SEQ ID NO: 17 - RNA, 19 nt, Artificial Sequence
  Whereas Artificial Sequence is comprised of siRNA.
ugacaaaggcaagaacguc

SEQ ID NO: 18 - RNA, 19 nt, Artificial Sequence
  Whereas Artificial Sequence is comprised of siRNA.
guuggguuuucuccauuca

SEQ ID NO: 19 - DNA, 33 bp, Artificial Sequence
  Whereas Artificial Sequence is comprised of DNAzyme.
tgattcctaggctagctacaacgatcctcgccg

SEQ ID NO: 20 - DNA, 33 bp, Artificial Sequence
  Whereas Artificial Sequence is comprised of DNAzyme.
cagcgcgaaggctagctacaacgacccagcagc

SEQ ID NO: 21 - DNA, 33 bp, Artificial Sequence
  Whereas Artificial Sequence is comprised of DNAzyme.
tgaggagtaggctagctacaacgacttggagcc

SEQ ID NO: 22 - DNA, 33 bp, Artificial Sequence
  Whereas Artificial Sequence is comprised of DNAzyme.
tcagcaccaggctagctacaacgagcccccctt

SEQ ID NO: 23 - DNA, 33 bp, Artificial Sequence
  Whereas Artificial Sequence is comprised of DNAzyme.
ggagctggaggctagctacaacgagttggccgt

SEQ ID NO: 24 - DNA, 33 bp, Artificial Sequence
  Whereas Artificial Sequence is comprised of DNAzyme.
cctcgttgaggctagctacaacgagtgttctga

SEQ ID NO: 25 - DNA, 33 bp, Artificial Sequence
  Whereas Artificial Sequence is comprised of DNAzyme.
agtcagtgaggctagctacaacgacttgtacca

SEQ ID NO: 26 - DNA, 33 bp, Artificial Sequence
  Whereas Artificial Sequence is comprised of DNAzyme.
cggcctccaggctagctacaacgagttcaggtt

SEQ ID NO: 27 - DNA, 33 bp, Artificial Sequence
  Whereas Artificial Sequence is comprised of DNAzyme.
ggagcgtgaggctagctacaacgagatggcctg

SEQ ID NO: 28 - DNA, 33 bp, Artificial Sequence
  Whereas Artificial Sequence is comprised of DNAzyme.
gcaccagcaggctagctacaacgactcagccac

SEQ ID NO: 29 - DNA, 33 bp, Artificial Sequence
  Whereas Artificial Sequence is comprised of DNAzyme.
cctttgtcaggctagctacaacgatctggtgct

SEQ ID NO: 30 - DNA, 33 bp, Artificial Sequence
  Whereas Artificial Sequence is comprised of DNAzyme.
ctcgggccaggctagctacaacgactgcctcag

SEQ ID NO: 31 - DNA, 33 bp, Artificial Sequence
  Whereas Artificial Sequence is comprised of DNAzyme.
gggaatctaggctagctacaacgaggggtgggt

SEQ ID NO: 32 - DNA, 33 bp, Artificial Sequence
  Whereas Artificial Sequence is comprised of DNAzyme.
cctaaggaaggctagctacaacgaagaatcctg

SEQ ID NO: 33 - DNA, 33 bp, Artificial Sequence
  Whereas Artificial Sequence is comprised of DNAzyme.
cggccatgaggctagctacaacgatcagaccca

SEQ ID NO: 34 - DNA, 33 bp, Artificial Sequence
  Whereas Artificial Sequence is comprised of DNAzyme.
gggcaaggaggctagctacaacgactggcactg

SEQ ID NO: 35 - DNA, 33 bp, Artificial Sequence
  Whereas Artificial Sequence is comprised of DNAzyme.
agtcgaacaggctagctacaacgaagacccgtg

SEQ ID NO: 36 - DNA, 33 bp, Artificial Sequence
  Whereas Artificial Sequence is comprised of DNAzyme.
aggctgtgaggctagctacaacgaggggtcgcc

SEQ ID NO: 37 - DNA, 33 bp, Artificial Sequence
  Whereas Artificial Sequence is comprised of DNAzyme.
gcccccggaggctagctacaacgagcacagaca

SEQ ID NO: 38 - DNA, 33 bp, Artificial Sequence
  Whereas Artificial Sequence is comprised of DNAzyme.
ctggtgctaggctagctacaacgaagagagccg

SEQ ID NO: 39 - DNA, 33 bp, Artificial Sequence
  Whereas Artificial Sequence is comprised of DNAzyme.
ccttcagcaggctagctacaacgacacgccccc

SEQ ID NO: 40 - DNA, 33 bp, Artificial Sequence
  Whereas Artificial Sequence is comprised of DNAzyme.
cggagtccaggctagctacaacgacttgaactc

SEQ ID NO: 41 - DNA, 33 bp, Artificial Sequence
  Whereas Artificial Sequence is comprised of DNAzyme.
cggccttcaggctagctacaacgatctgggagg

SEQ ID NO: 42 - DNA, 33 bp, Artificial Sequence
  Whereas Artificial Sequence is comprised of DNAzyme.
cagccacgaggctagctacaacgagcccaggaa

SEQ ID NO: 43 - DNA, 33 bp, Artificial Sequence
  Whereas Artificial Sequence is comprised of DNAzyme.
gcaggagtaggctagctacaacgatctccccac

SEQ ID NO: 44 - DNA, 33 bp, Artificial Sequence
  Whereas Artificial Sequence is comprised of DNAzyme.
gcgctgtcaggctagctacaacgatcaaggagc

SEQ ID NO: 45 - DNA, 33 bp, Artificial Sequence
  Whereas Artificial Sequence is comprised of DNAzyme.
gccctgtgaggctagctacaacgactctgtggc

SEQ ID NO: 46 - DNA, 33 bp, Artificial Sequence
  Whereas Artificial Sequence is comprised of DNAzyme.
tagctctgaggctagctacaacgacggccctgc

SEQ ID NO: 47 - DNA, 33 bp, Artificial Sequence
  Whereas Artificial Sequence is comprised of DNAzyme.
acttgcagaggctagctacaacgacagcatggc

SEQ ID NO: 48 - DNA, 33 bp, Artificial Sequence
  Whereas Artificial Sequence is comprised of DNAzyme.
tgaccagcaggctagctacaacgacagcacctc

SEQ ID NO: 49 - DNA, 33 bp, Artificial Sequence; identical to SEQ ID NO: 21
  Whereas Artificial Sequence is comprised of DNAzyme.

SEQ ID NO: 50 - DNA, 33 bp, Artificial Sequence
  Whereas Artificial Sequence is comprised of DNAzyme.
ccgtgcccaggctagctacaacgagggctcggg

SEQ ID NO: 51 - DNA, 33 bp, Artificial Sequence; identical to SEQ ID NO: 24
  Whereas Artificial Sequence is comprised of DNAzyme.

SEQ ID NO: 52 - DNA, 33 bp, Artificial Sequence
  Whereas Artificial Sequence is comprised of DNAzyme.
agaccagcaggctagctacaacgaggccgtctc

SEQ ID NO: 53 - DNA, 33 bp, Artificial Sequence; identical to SEQ ID NO: 33
  Whereas Artificial Sequence is comprised of DNAzyme.

SEQ ID NO: 54 - DNA, 34 bp, Artificial Sequence
  Whereas Artificial Sequence is comprised of DNAzyme.
gccctgtgaggctagctacaacgactctgtggct

SEQ ID NO: 55 - DNA, 34 bp, Artificial Sequence
  Whereas Artificial Sequence is comprised of DNAzyme.
gccctgtgaggctagctacaacgactctgtggcg

SEQ ID NO: 56 - DNA, 34 bp, Artificial Sequence
  Whereas Artificial Sequence is comprised of DNAzyme.
gccctgtgaggctagctacaacgactctgtggcc

SEQ ID NO: 57 - DNA, 34 bp, Artificial Sequence; identical to SEQ ID NO: 54
  Whereas Artificial Sequence is comprised of DNAzyme.

SEQ ID NO: 58 - DNA, 34 bp, Artificial Sequence; identical to SEQ ID NO: 55
  Whereas Artificial Sequence is comprised of DNAzyme.